(19) United States
(12) Patent Application Publication
(10) Pub. No.: US 2026/0114886 A1
(43) Pub. Date: Apr. 30, 2026

(54) CONTROL OF IVL SYSTEMS, DEVICES AND METHODS THEREOF

(71) Applicant: Cardiovascular Systems, Inc., Santa Clara, CA (US)

(72) Inventors: Gregory B. Ingersoll, Minneapolis, MN (US); Jason W. Staab, Apple Valley, MN (US); Austin P. Petronack, Plymouth, MN (US); John R. Ballard, Waconia, MN (US); J. Samuel Batchelder, Woodinville, WA (US); Jacob T. Williams, Plymouth, MN (US); Donald D. Hanson, Shakopee, MN (US); Thomas D. Brindley, Plymouth, MN (US); Scott P. Boeshart, Plymouth, MN (US); Jeffrey T. Moriarty, Roseville, MN (US)

(21) Appl. No.: 19/431,242

(22) Filed: Dec. 23, 2025

Related U.S. Application Data
(63) Continuation-in-part of application No. 19/170,999, filed on Apr. 4, 2025.
Application No. 19/170,999 is a continuation of application No. PCT/US2023/079209, filed on Nov. 9, 2023.
(60) Provisional application No. 63/424,573, filed on Nov. 11, 2022.
(60) Provisional application No. 63/580,547, filed on Sep. 5, 2023.
(60) Provisional application No. 63/874,995, filed on Sep. 3, 2025.

(51) Int. Cl. A61B 17/22; A61B 17/00

(52) U.S. Cl. CPC A61B 17/22022; A61B 2017/00199; A61B 2017/00477; A61B 2017/22025; A61B 2017/22062

(57) ABSTRACT

An intravascular lithotripsy (IVL) console is disclosed, comprising a power plug configured to couple to a mains power source, with the power plug extending outside an enclosure housing internal console circuitry. The console includes conductors coupled to the power plug and a first isolation barrier that electrically isolates power from the mains power source. The internal console circuitry includes low-voltage control circuitry and high-voltage power circuitry, which generates high-voltage pulses for emitters in a catheter. A second isolation barrier couples a first processor in the low-voltage control circuitry to a second processor in the high-voltage power circuitry. The second processor receives commands from the first processor and causes the high-voltage power circuitry to deliver high-voltage pulses to the emitters. The isolation barriers allow the power plug to supply power while the high-voltage power circuitry delivers pulses to the emitters.

### CROSS-REFERENCE TO RELATED APPLICATIONS

This application is a continuation in part of application Ser. No. 19/170,999, filed Apr. 4, 2025, entitled “CONTROL OF IVL SYSTEMS, DEVICES AND METHODS THEREOF”, which is a continuation of PCT/US2023/079209, filed Nov. 9, 2023, entitled “CONTROL OF IVL SYSTEMS, DEVICES AND METHODS THEREOF”, which claims benefit of provisional application No. 63/424,573, filed Nov. 11, 2022, entitled “DEVICES, SYSTEMS, AND METHODS OF INTRAVASCULAR LITHOTRIPSY”, and provisional application No. 63/580,547, filed Sep. 5, 2023, entitled “DEVICES, SYSTEMS AND METHODS OF INTRAVASCULAR LITHOTRIPSY” and provisional application No. 63/874,995, filed Sep. 3, 2025, entitled “HIGH-VOLTAGE/LOW-VOLTAGE ISOLATION IN IVL DEVICES”, the entire contents of each of which is incorporated herein by reference.

### BACKGROUND

Intravascular lithotripsy (IVL) is an advanced medical procedure utilizing high-energy pulses to break apart calcified plaque within blood vessels, restoring vessel patency and improving patient outcomes. The IVL console acts as the control and power center for these pulses, traditionally requiring a reliable energy source to generate the high voltages used in therapy.

Historically, directly connecting a console to a hospital's mains power supply presented significant risks for both patients and clinical operators. Medical devices that interface with a patient's body—especially those delivering high-voltage pulses—can be vulnerable to electrical hazards if isolation between the mains supply and patient-contacting circuitry is insufficient. Failure to achieve robust isolation may result in leakage currents, which—even at very low levels—can unintentionally stimulate patient tissue, causing discomfort, injury, or even adverse physiological effects such as arrhythmias. Likewise, compromised isolation puts operators at risk of exposure to hazardous voltages, potentially leading to electric shock, equipment damage, or interruption of critical interventions.

Due to the complexity and limitations of achieving complete electrical isolation when using mains power, most IVL consoles are now designed to operate solely on battery power. This approach effectively eliminates the problems associated with direct connection to external power sources, dramatically reducing the risk of leakage currents or accidental voltage exposures for both patients and clinicians. By relying on battery operation, the system avoids the technical hurdles and safety concerns that arise from isolation barriers, simplifying compliance with regulatory standards such as IEC 60601, and fostering a safer clinical environment.

Even with battery power systems need to maintain separation between the low-voltage control circuitry (which handles operator inputs and logic) and the high-voltage power circuitry (responsible for generating therapeutic pulses). Robust isolation within the console architecture is used to prevent high voltages from reaching user interfaces or external equipment, ensuring consistent safety for all involved.

In summary, current ILV systems implement battery-only operation to address the risks of electrical hazards, streamline device architecture, and deliver enhanced protection for both patients and healthcare professionals during intravascular lithotripsy procedures. However, this battery-only approach results in limitations on the time and amount of therapy that can be delivered by an IVL console.

The subject matter claimed herein is not limited to embodiments that solve any disadvantages or that operate only in environments such as those described above. Rather, this background is only provided to illustrate one exemplary technology area where some embodiments described herein may be practiced.

### BRIEF DESCRIPTION OF THE SEVERAL VIEWS OF THE DRAWINGS

These drawings are exemplary illustrations of certain embodiments and, as such, are not intended to limit the disclosure.

FIG. 1 illustrates a diagrammatic intravascular lithotripsy (IVL) arrangement according to one or more embodiments of the present disclosure.

FIG. 2 illustrates two emitters defined within a support body.

FIG. 3 illustrates additional details with respect to low-voltage and high-voltage isolation in the voltage pulse generator console.

FIG. 4 is a flowchart of an example method for operating an intravascular lithotripsy (IVL) console.

FIG. 5 illustrates a control operation flow diagram applicable to the IVL arrangement of FIG. 1 according to one or more embodiments of the present disclosure.

FIG. 6A illustrates portions of circuitry arrangements of the IVL arrangement of FIG. 1 according to one or more embodiments of the present disclosure.

FIG. 6B illustrates portions of circuitry arrangements of the IVL arrangement of FIG. 1 according to one or more embodiments of the present disclosure.

FIG. 7A illustrates additional portions of circuitry arrangements of the IVL arrangement of FIG. 1 according to one or more embodiments of the present disclosure.

FIG. 7B illustrates additional portions of circuitry arrangements of the IVL arrangement of FIG. 1 according to one or more embodiments of the present disclosure.

FIG. 7C illustrates additional portions of circuitry arrangements of the IVL arrangement of FIG. 1 according to one or more embodiments of the present disclosure.

FIG. 7D illustrates additional portions of circuitry arrangements of the IVL arrangement of FIG. 1 according to one or more embodiments of the present disclosure.

FIG. 8 illustrates a flowchart for controlling and delivering voltage to electrodes and generating shock waves according to one or more embodiments of the present disclosure.

FIG. 9 illustrates a control operation flow applicable to the IVL arrangement of FIG. 1 according to one or more embodiments of the present disclosure

FIG. 10 illustrates graphic comparisons of voltages applied to a known IVL device and to embodiments of IVL devices according to the present disclosure.

FIG. 11 illustrates a graphic comparison of average peak pressure generated over 80 voltage pulses by a known IVL devices and embodiments of IVL devices according to the present disclosure.

FIG. 12 illustrates a graphic comparison of average peak pressure generated over 80 voltage pulses by a known IVL device and over a predetermined maximum number of voltage pulses by embodiments of IVL devices according to the present disclosure.

FIG. 13 illustrates an exemplary flowchart method according to the present disclosure.

### DETAILED DESCRIPTION OF THE INVENTION

The present disclosure pertains to an improved intravascular lithotripsy (“IVL”) console, specifically directed to systems and methods for electrically isolating patient-contacting medical equipment from hazardous voltages while enabling reliable operation from a mains power source. To address risks of electrical leakage currents and compromised isolation between patient-facing and/or operator-facing components and external energy supplies—the present disclosure introduces a console architecture that incorporates layered isolation barriers to ensure optimal safety and performance.

The disclosed IVL console comprises a power plug configured for direct coupling to a mains power source, with one or more conductors extending from the plug to the console's internal circuitry. A first isolation barrier is positioned to electrically isolate incoming power from sensitive console components according to a predetermined dielectric strength. For example, this isolation may be part of a transformer or other power supply isolation. This isolation is configured to have a certain predetermined voltage dielectric strength. This barrier mitigates electrical hazards by preventing leakage currents and/or high-voltage to low-voltage circuit breakdown and protecting both patients and clinical operators during therapeutic delivery.

Within a console enclosure, the console is further divided into low-voltage control circuitry and high-voltage power circuitry. The high-voltage circuitry serves to generate pulses for intravascular therapy, while the low-voltage control section governs user input and operational commands. A second isolation barrier is provided between these two domains. The second isolation barrier couples a first processor (within the control circuitry) to a second processor (within the high-voltage circuitry). This configuration ensures that control commands are safely relayed without direct electrical connection, leveraging magnetic or optical coupling methods to achieve robust isolation.

The dual-barrier approach as described herein enables the IVL console to maintain a secure connection to mains power, supplying necessary energy to all functional units even during delivery of high-voltage therapeutic pulses to emitters in an associated catheter. The console architecture is further adaptable to embodiments involving expandable balloons with multiple electrodes for spark generation, expanding the versatility and clinical utility of the system.

By implementing these enhanced isolation strategies, embodiments address the limitations of battery-only designs, paving the way for longer, uninterrupted therapy sessions without compromising safety. Further, embodiments may facilitate the ability to perform more consecutive procedure sessions with different patients without delaying to recharge the console. The following detailed description elaborates upon the technical aspects and operational modes of various embodiments.

Traditional intravascular lithotripsy (IVL) devices, systems, and methods can apply high-energy electrical power to generate a spark (arc) between discharge electrodes. Under appropriate conditions, sparks generated by submerged electrodes can generate pressure waves within the medium, which can be applied to treat (breakup) calcified lesions within the patient's vasculature. It can be appreciated that appropriate control of such high-energy systems can be paramount to effective treatment, including safe treatment.

Intravascular lithotripsy systems, devices and methods have been described by Applicant. See PCT/2022/074607, filed Aug. 5, 2022 and entitled “INTRAVASCULAR LITHOTRIPSY BALLOON SYSTEMS, DEVICES AND METHODS”, the entire contents of which are hereby incorporated by reference.

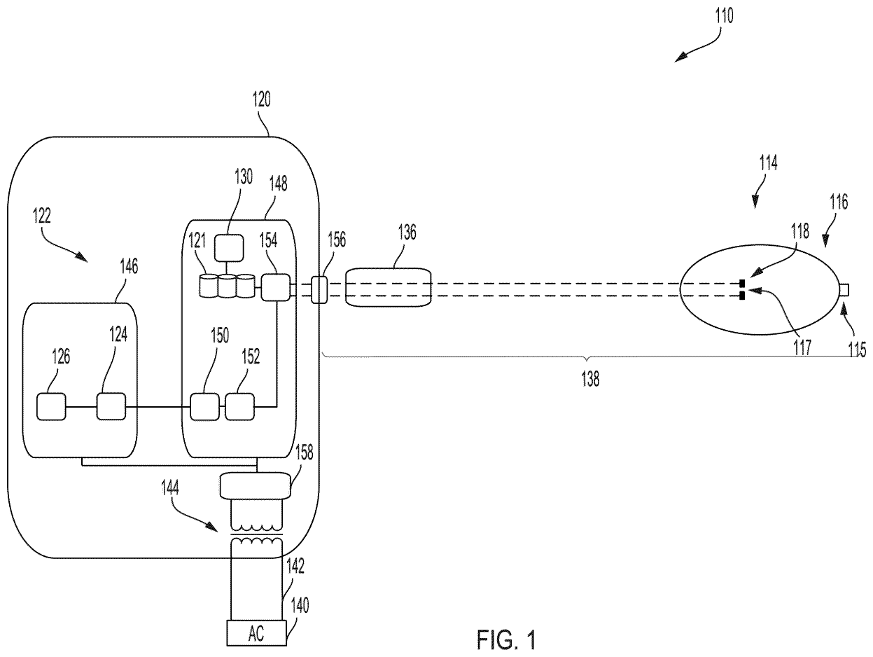

As illustrated in FIG. 1, a diagrammatic or schematic layout of portions of an exemplary IVL system 110 is provided. The illustrative IVL system 110 comprises a voltage pulse generator console 120 and a catheter assembly 114. Generally, the voltage pulse generator console 120 provides high-voltage pulses to the catheter assembly 114 to perform IVL therapy.

The catheter assembly 114 includes an elongate body, embodied as a catheter having guidewire 115, and a fluid-filled member 116 configured to contain conductive fluid therein, exemplified by an inflatable balloon, disposed near the distal end of the elongate body and arranged to receive fluid for inflation to facilitate IVL therapy. A set of dischargeable spaced-apart electrodes 118 are shown arranged within the exemplary fluid-filled member 116, at least some of which are spaced-apart by a gap 117 from each other to facilitate creation of a spark or electrical arc between the set of spaced-apart electrodes 118.

The IVL system embodiments described herein may be used in connection with electrodes that are within a fluid-filled member 116 configured to contain a fluid, e.g., a conductive fluid, therein. The fluid-filled member 116 embodiments may include an inflatable balloon or enclosure as shown in FIG. 1, which may be compliant or non-compliant and serves to contain the fluid such that the spaced-apart electrodes 118 are preferably fully submerged within the contained fluid. In addition, the fluid-filled member 116 may comprise a fillable member that is at least partially rigid and/or not flexible. In other embodiments, the fluid-filled member 116 may contain the fluid therein, and wherein the set of spaced-apart electrodes 118 is fully submerged within the contained fluid during the generation of an electrical arc between the spaced-apart electrodes during an IVL procedure.

Alternatively, the IVL system control embodiments of the present disclosure may be used in connection with electrodes that are not located or surrounded by a fluid-filled or fillable member 116. In these embodiments, the IVL system may comprise one or more sets of spaced-apart electrodes 118 that may be continuously or periodically exposed to saline or other fluid, and during the exposure, the IVL system may generate an electrical arc between the spaced-apart electrodes 118.

Each of the spaced-apart electrodes in FIG. 1 is arranged in communication (as suggested by dashed line conductors) with an electric pulse generation system, or voltage pulse generator console 120 to receive high-voltage electrical energy for spark generation to create pressure waves for IVL therapy. In the example illustrated, the catheter assembly 114 is coupled to the voltage pulse generator console 120 through a connector 156 which allows for selectively coupling the catheter assembly to the voltage pulse generator console 120. In the illustrative embodiment, one electrode may be grounded and the other provided with high-voltage from the voltage pulse generator console 120, although in some embodiments, any voltage differential may be applied. The voltage pulse generator console 120 may include an IVL control system 122 which includes high-voltage power circuitry 148 and other circuitry to control delivery of high-voltage pulses from the high-voltage power circuitry 148.

In the example illustrated, the voltage pulse generator console 120 is powered by a power plug 140 being coupled to mains power via conductors 142 external to the voltage pulse generator console 120. Power is supplied from the mains power through a first isolation barrier 144, that in this example is implemented as a transformer. Power is provided from the first isolation barrier 144 to a battery 158. The battery 158 provides power to the low-voltage control circuitry 146 and the high-voltage power circuitry 148. The first isolation barrier 144 isolates the mains power from the patient and operator as power supplied to the patient is supplied from the high-voltage power circuitry 148 and power used by the operator is supplied from the low-voltage control circuitry 146 and high-voltage power circuitry 148. For example, the first isolation barrier 144 may isolate circuitry, operators, and/or patients against an AC power source fault.

In an alternative embodiment, a power path controller is used where if mains power is connected, the input power splits to charge the battery and power the system. The system input becomes a logical OR of mains AC/DC or battery rather than always using the battery (which may or may not be charging at the same time). This embodiment can be useful in cases where the battery malfunctions and/or if the system utilizes a removable/exchangeable battery pack. In this case, if the system is plugged into mains power, the system will continue to operate whether the battery is installed or not.

Typically, the high-voltage power circuitry 148 is implemented on a circuit board that is separate from the low-voltage control circuitry 146 that will be discussed in more detail below. The high-voltage power circuitry 148 includes a capacitor bank 121 coupled to the catheter assembly 114 through a switch 154. The switch may be implemented using a reed relay, a high power IGBT, spark gap switch, combinations thereof or other high power switching component(s). The switch 154 is controllable to deliver a high-voltage pulse to the spaced-apart electrodes 118 from energy stored on the capacitor bank 121 or other high-voltage source.

The IVL control system 122 further includes low-voltage control circuitry 146. The low-voltage control circuitry 146, as noted above, is typically implemented on a different circuit board than the high-voltage power circuitry 148. In particular, this may be part of an isolation configuration to isolate the low-voltage control circuitry 146 from the high-voltage power circuitry 148. The low-voltage control circuitry 146 comprises a first processor 124 configured for executing instructions stored on memory 126 to provide communications signals through a second isolation barrier 150 to a second processor 152 in the high-voltage power circuitry 148 for IVL operations according to the first processor governance. In some embodiments, the first processor comprises part number MK50DN512CLL10 and the second processor comprises part number MKL81Z128VMC7 both available from NXP USA Inc. of Austin, TX. In some embodiments, the second isolation barrier 150 may include electronic and/or magnetic isolators. Magnetic isolators may use, for example, inductive devices and/or transformers. For example, in some embodiments, the isolation barrier may include a magnetic signal digital isolator such as part number ADUM4401 available from Analog Devices of Wilmington, Massachusetts. In an alternative or additional embodiment, the isolation barrier may include an optical digital signal isolator. This isolation scheme can isolate high voltages from the low-voltage control circuitry 146 thus providing protection to a device operator from the high voltages.

In an illustrative example, the first processor 124 can execute instructions stored on the memory 126 according to a designed pulse sequence or individual pulse. Note that the memory 126 may be part of the first processor 124 or implemented separately from the first processor 124. The first processor 124 can provide signal instructions to the second processor 152 through the isolation barrier 150. The second processor 152 then executes instructions at the second processor 152 [stored on memory of the second processor 152 and/or in external memory (not shown)] to cause the switch 154 to deliver voltage pulses from energy stored on the capacitor bank 121 to the spaced-apart electrodes 118.

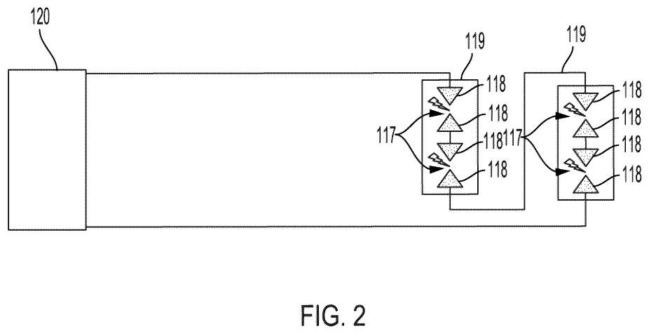

An exemplary IVL system is shown in FIG. 2, illustrating one method for applying voltage pulses to the system, resulting in current flow through the exemplary system to each set of spaced-apart electrodes 118, where each set of spaced-apart electrodes 118 forms a spark gap 117. In the illustrated example, two sets of spaced-apart electrodes form an emitter 119. A given emitter 119 may include two spark gaps so as to accomplish more complete energy coverage, such as when the spark gaps are located on conductive bands around a catheter lumen to allow for pressure waves in multiple directions. Note that in other embodiments, emitters may include a single spark gap or more than two spark gaps. The emitters 119, and thus the four sets of spaced-apart electrodes (and thus spark gaps 117) are connected in series. FIG. 2 illustrates two emitters 119 defined within a support body. Application of a voltage pulse of sufficient magnitude and/or duration from the voltage pulse generator console 120 to the sets of serially connected spark gaps 117 will result in a production of electrical arcs as follows: a first arc across a first spark gap; a second arc across a second spark gap, a third arc across a third spark gap and a fourth arc across a fourth spark gap.

Appropriate control of such high-energy systems can also require achieving sufficient energy at the discharge site to create an electrical arc across the one or more spark gaps of an IVL system. Embodiments of the IVL systems, devices and methods described within the present disclosure may include operation for adjusting the total electrical energy provided to the system at each emitter. Such control systems for intravascular lithotripsy systems, devices and methods have been described by Applicant. See PCT/2023/079209, filed by Applicant on Nov. 9, 2023 and entitled “CONTROL OF IVL SYSTEMS, DEVICES AND METHODS THEREOF”; U.S. Ser. No. 18/290,173, filed by Applicant on Nov. 10, 2023 and entitled “CONTROL OF IVL SYSTEMS, DEVICES AND METHODS THEREOF”; U.S. Ser. No. 18/506,339, filed by Applicant on Nov. 10, 2023 and entitled “CONTROL OF IVL SYSTEMS, DEVICES AND METHODS THEREOF”; U.S. Ser. No. 18/506,416, filed by Applicant on Nov. 10, 2023 and entitled “CONTROL OF IVL SYSTEMS, DEVICES AND METHODS THEREOF”; and U.S. Ser. No. 18/506,428, filed by Applicant on Nov. 10, 2023 and entitled “CONTROL OF IVL SYSTEMS, DEVICES AND METHODS THEREOF”, the entire contents of which are hereby incorporated by reference. The voltage pulse may be generated by discharging energy stored in the voltage pulse generator console 120, and in particular, in some embodiments, discharging energy stored in the voltage pulse generator console 120, including potentially the capacitor bank 121 (FIG. 1) which may produce an electrical arc across a spark gap between two spaced-apart electrodes. In some embodiments, the voltage pulses may be generated in one or more series of voltage pulses, wherein each adjacent series of voltage pulses may be spaced-apart or separated by time or other mechanism.

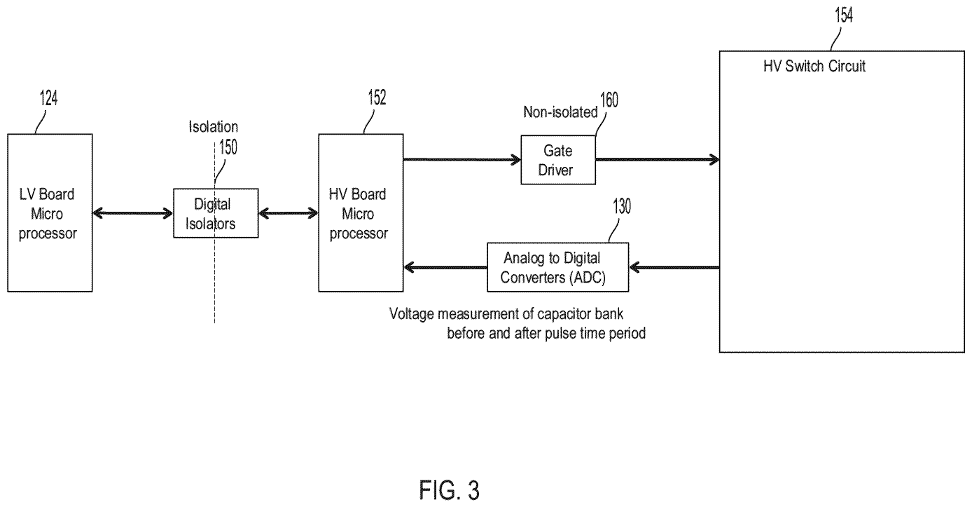

FIG. 3 illustrates additional details with respect to low-voltage and high-voltage isolation in the voltage pulse generator console 120. As illustrated previously, the first processor 124 communicates instructions to the second processor 152 through the second isolation barrier 150. Note that in some embodiments, the first processor 124 may send an executable instruction comprising commands and arguments to the second processor 152. Alternatively, the first processor 124 may send a data packet to the second processor 152. In yet another example, the first processor 124 may simply send a single bit or multibit flag to the second processor 152. In one particular example, the second isolation barrier 150 includes a digital isolator as described previously. Such devices may include, for example, complementary high-voltage and low-voltage circuits implementing a clock input, a chip select input, a serial peripheral interface (SPI) input and an SPI output. In this case, typically data and/or instructions would be provided across the second isolation barrier 150 using such a device.

The second processor 152, in response to communication from the first processor 124 performs programmed actions to produce outputs for controlling the switch 154. In some embodiments, this may include determining to close the switch 154 to provide a high-voltage pulse from the capacitor bank 121 to a set of spaced-apart electrodes 118 to produce a spark. In some embodiments, this may be accomplished by driving an output pin on the second processor 152 high (or low depending on whether positive or negative logic is used) to drive a gate driver 160 to provide sufficient power to close the switch 154.

Note that while a single output pin from the second processor 152, a single gate driver 160, and a single switch 154 are shown, it should be appreciated that in other embodiments, a plurality of one or more of each of these may be present so as to drive emitter circuits independently from one another. In particular, if a plurality of spark gaps 117 and/or emitter 119 are implemented in a catheter assembly 114, at least some of these can be controlled independently of others by using different output pins, gate drivers, and relays. For example, in one embodiment, one or more common gate drivers are used, but are used in conjunction with a plurality of one or more reed relay sets and a plurality of output pins to allow for driving different emitters or sets of spaced-apart electrodes. Thus, the voltage pulse generator console 120 may be in operative and electrical association with one or more sets of spaced-apart electrodes 118. As illustrated, in some embodiments, such as that illustrated in FIG. 2, the various spaced-apart electrodes are connected in a serial electrical connection with the voltage pulse generator console 120 operatively and/or electrically connected with a more proximally located emitter 119. Alternative connection associations between the set(s) of spaced-apart electrodes 118 or emitter(s) 119 and the voltage pulse generator console 120 may be provided in alternative embodiments. For example, the two sets of spaced-apart electrodes may be connected in a parallel arrangement. Further, the voltage pulse generator console 120 may be operatively and/or electrically associated with a more distal spaced-apart set of electrodes 118 or emitter 119. Further different sets of electrodes 118 and/or different emitters 119 may be controlled independently.

FIG. 3 further illustrates a monitor 130 that may monitor voltages and/or currents. In some embodiments, the monitor is coupled directly to the capacitor bank 121 as shown in FIG. 1, while in other embodiments the monitor 130 is coupled to other components, such as the switch 154 to monitor voltages and/or currents delivered to emitters and/or residual voltages on the capacitor bank 121 after a spark has been delivered. In still other alternative embodiments, the monitor 130 may be located external to the voltage pulse generator console 120. The second processor 152 is in operative and/or electrical association with a monitor 130. The second processor 152 can provide information about voltages and/or currents monitored by the monitor 130 through the second isolation barrier 150 to the first processor 124, where such information can be used for reporting, spark control, or other purposes. In some embodiments, the monitor 130 may be located in a separate location apart from the voltage pulse generator console 120.

The system's architecture is designed to ensure both safety and operational efficiency. Redundant isolation strategies, including both physical and electrical barriers, are integrated to defend against possible faults or cross-domain interference. Various sections of circuitry—whether low or high voltage—are physically partitioned within an enclosure, minimizing (by shielding) electromagnetic interference and preventing accidental contact with hazardous voltages.

Embodiments may include a display mechanism, such as an LCD or LED panel, configured to provide real-time feedback on system status, operating parameters, and error notifications for enhanced procedural safety. Some embodiments may include one or more status indicators, such as visual LEDs or audible alerts, to signify operational states-including readiness, pulse delivery, fault, or standby-thereby facilitating intuitive user interaction.

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

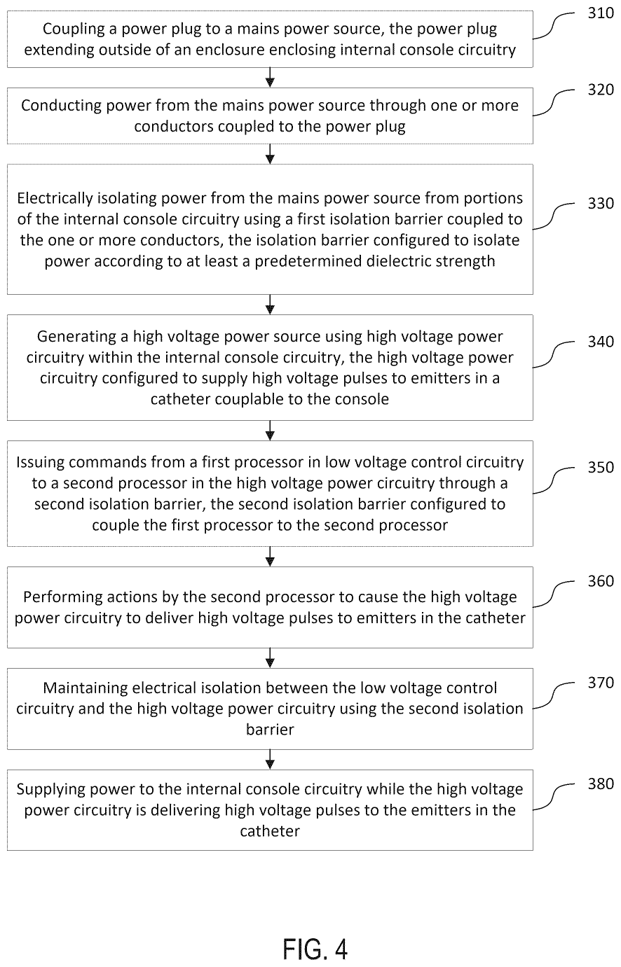

FIG. 4 is a flowchart of an example method operating an intravascular lithotripsy (IVL) console.

At step 310, the method includes coupling a power plug to a mains power source, the power plug extending outside of an enclosure enclosing internal console circuitry.

At step 320, the method includes conducting power from the mains power source through one or more conductors coupled to the power plug.

At step 330, the method includes electrically isolating power from the mains power source from portions of the internal console circuitry using a first isolation barrier coupled to the one or more conductors, the isolation barrier configured to isolate power according to at least a predetermined dielectric strength.

At step 340, the method includes generating a high-voltage power source using high-voltage power circuitry within the internal console circuitry, the high-voltage power circuitry configured to supply high-voltage pulses to emitters in a catheter couplable to the console]

At step 350, the method includes issuing commands from a first processor in low-voltage control circuitry to a second processor in the high-voltage power circuitry through a second isolation barrier, the second isolation barrier configured to couple the first processor to the second processor.

At step 360, the method includes performing actions by the second processor to cause the high-voltage power circuitry to deliver high voltage pulses to emitters in the catheter.

At step 370, the method includes maintaining electrical isolation between the low-voltage control circuitry and the high-voltage power circuitry using the second isolation barrier.

At step 380, the method includes supplying power to the internal console circuitry while the high-voltage power circuitry is delivering high voltage pulses to the emitters in the catheter.

Further, the methods may be practiced by a computer system including one or more processors and computer-readable media such as computer memory. In particular, the computer memory may store computer-executable instructions that when executed by one or more processors cause various functions to be performed, such as the acts recited in the embodiments.

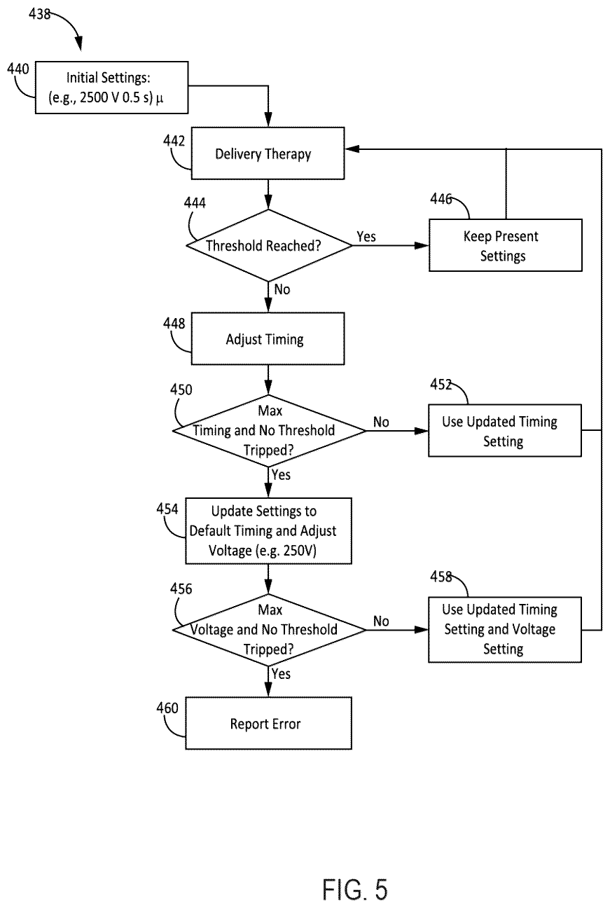

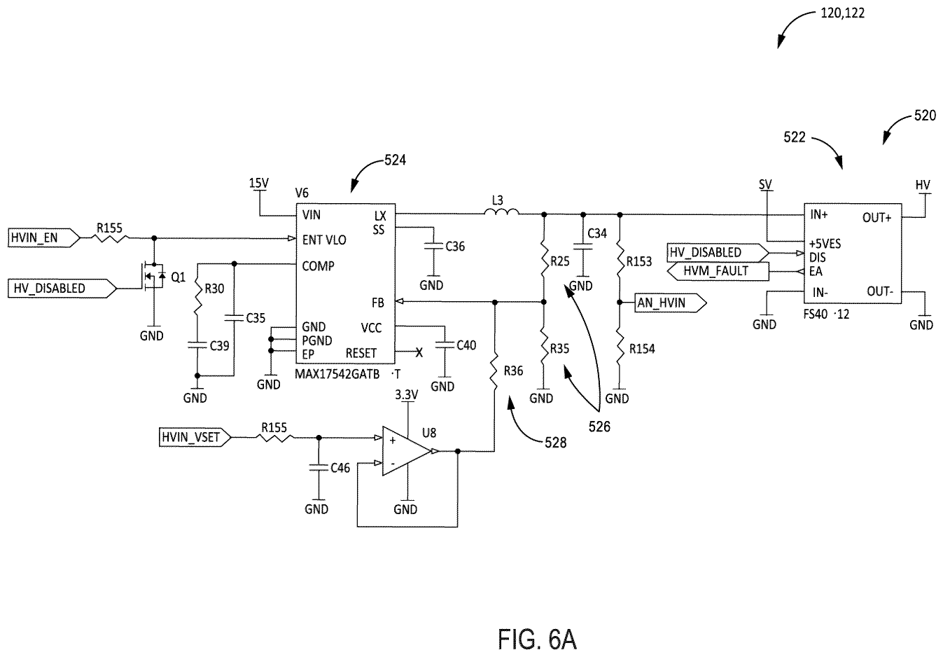

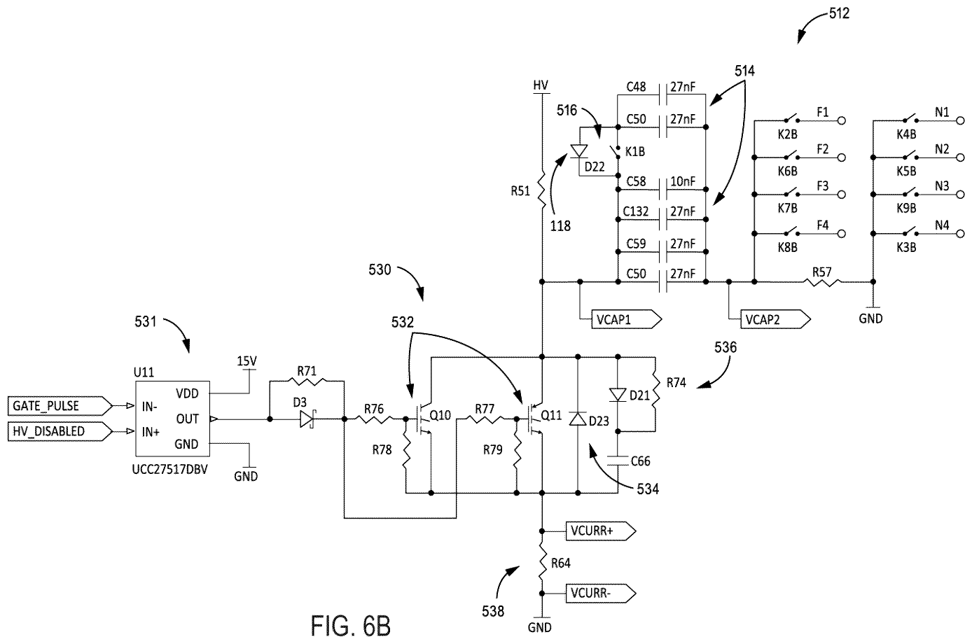

Referring now to FIG. 5, a flow diagram is shown concerning one embodiment of a control 438 in operation of IVL as discussed concerning boxes 440 through 460. Such control operations can be governed by the electric pulse generation system 20, and illustratively by the IVL control system 122 (see FIG. 6A). As discussed in additional detail herein, control 438 applies incremental changes in power parameters during cycling of applied voltage while monitoring parameters related to spark generation. For example, incrementally increasing the duration of voltage applied to the discharge electrodes can increase the likelihood of generation of an effective spark without excessive energy.

Moreover, if increases to the duration of voltage applied to the discharge electrodes fails to generate sufficient spark, incremental increase to the voltage can further increase the likelihood of generation of an effective spark without excessive power. Still further, if incrementally increased voltage fails to generate sufficient spark, duration can once again be incrementally increased before further increasing voltage. Accordingly, it can be appreciated that controlled incremental increases in duration and voltage can be implemented to achieve effective spark generation, and thus pressure wave generation, at or near the lowest required power characteristics for effective spark generation. Increasing the likelihood of reaching sufficient spark with lower power can increase the efficiency, safety, and/or reduce intensity of effective IVL therapy.

In box 440, initial settings are applied. For illustrative example, default initial settings are applied as a discharge voltage of 2500 volts (V) for a duration of 0.5 microseconds. In some embodiments, the initial settings may be determined by any suitable manner, including by use of programmable defaults, as adjusted settings based on use, for example, adjusted based on the patient characteristics, environmental conditions, procedural approach, and/or product cycle lifetime (i.e., operable age), among other aspects. From box 440, control may proceed to box 442.

In box 442, electrical energy is applied to the electrodes to deliver IVL therapy. In the first instance of proceeding from box 440 to box 442, electrical energy is applied at the initial settings, e.g., an applied voltage of 2500 V for a duration of 0.5 microseconds. When applied in a clinical setting, such that the IVL catheter is arranged within the patient's body lumen, and specifically with discharge electrodes submerged within a fluid medium within a fluid-filled member such as an angioplasty balloon, pressure wave therapy can ensue. However, as mentioned below, at present conditions, in some instances the energy provided at the initial settings may be insufficient to generate a spark at the electrodes, or may generate insufficient spark or insufficient pressure wave. From box 442, control may proceed to box 444.

In box 444, determination of threshold aspects is conducted. In the illustrative embodiment, a determined value for current applied in box 442 is compared with threshold current value. In the illustrative example, the threshold current value is embodied as a predetermined fixed value, e.g., 20 amperes (amps), but in some embodiments, may have any suitable value, for example 50 amps, 100 amps, 150 amps, 175 amps, the threshold current value for a given cycle may be determined based on the number of previous cycles in a therapy session, the number of cycles maintaining present settings (e.g., through box 446) in a therapy session, the number of particular successive cycles (e.g., number of successive cycles through box 446 or box 448 or box 452 or box 454 or box 458), the patient characteristics, environmental conditions, procedural approach, and/or product cycle lifetime (i.e., operable age), among other aspects. Additionally, although the threshold current value may be set to one value, the actual current passed during a sufficient generation of arc across the electrodes may be greater.

Responsive to determination that the determined value for current applied in box 442 is equal to or greater than the threshold current value, control may proceed to box 446. Otherwise, responsive to determination that the determined value for current applied in box 442 less than the threshold current value, control may proceed to box 448. For example, as mentioned above, insufficient spark generation may result in little or no current flow (e.g., about 0 to about 5 amps, which may represent dissipated energy into the medium without arcing) across the electrodes, which would not achieve the threshold current value, and thus would proceed to box 448.

In the illustrative embodiment, the determined current value for current applied in box 442 is embodied as the instantaneous current applied across the electrodes. In some embodiments, the determined current value may be embodied as an aggregated value, such as a time-averaged value of current applied to the electrodes. Continuing from the exemplary embodiment applying a threshold current value of 20 amps, when 20 amps is achieved by the therapy delivered, the spark generation is deemed sufficient.

It can be appreciated that the substantial occurrence of 20 amps provides considerable current across the discharge electrodes indicating the generation of meaningful spark. By comparison little or no current may flow across the discharge electrode when a spark fails to generate or when insufficient spark is generated under the applied duration and at the applicable voltage, 2500 V by example, and which can generally range between about 500 V to about 5000 V for IVL therapy, although conceivably can range between about 100 V to about 10000 V in terms of practical application.

In box 446, determination is made to maintain presently selected settings. In the illustrative embodiment, the presently selected settings include the discharge voltage of and applicable duration as applied immediately previously in box 442. For avoidance of doubt, if the initial settings have just immediately been applied in box 442, resulting in 20 amps of current, the initial settings would be applied in the next cycle of therapy. However, if the presently selected settings include updates settings, for example, updated duration and/or voltage settings from later portions of control 438, as discussed in additional detail herein, then maintaining presently selected settings would include the immediately applied updated settings. Maintaining presently selected settings proceeds openly to return to box 442, to again apply electrical energy to the electrodes to deliver IVL therapy.

In box 448, determination is made to increase the duration of applied voltage to the discharge electrodes. Continuing the example in which less than 20 amps of current indicates insufficient spark generation, rather than immediately increasing the voltage applied, the duration of applied voltage can be incrementally increased. At the microsecond-scale, increasing the duration of applied voltage can increase the likelihood of sufficient spark generation using the same voltage previously applied. This can be achieved as the result of overcoming threshold system impendence and/or other factors affecting the ease of spark generation during a given cycle at a given voltage.

The duration of applied voltage is increased by a predetermined duration interval, illustratively embodied as a fixed value, e.g., 0.5 microseconds. In some embodiments, the predetermined interval for given cycle may be determined based on factors such as the number of times therapy cycles have ensued previously during the therapy session (e.g., number of cycle intervals have proceeding through boxes 442, 444, 446, prior to proceeding to box 448), the patient characteristics, environmental conditions, procedural approach, and/or product cycle lifetime (i.e., operable age), among other aspects. In some embodiments, the predetermined duration interval for a given cycle may be varied by predetermined rate of change, for example, by percentage gain or loss by cycle. Control proceeds openly from box 448 to box 450.

In box 450, determination is made whether maximum duration has been achieved. In the illustrative embodiment, the maximum duration is a predetermined duration embodied as a fixed value, e.g., 6 microseconds. By way of example, if the control sequence progresses through cycles from initial settings of box 440 at 0.5 microseconds through box 448, until reaching 6 microseconds, the maximum duration would be achieved as a threshold value.

In some embodiments, the maximum duration for a given cycle may be determined based on the number of previous cycles in a therapy session, the number of cycles maintaining present settings (e.g., through box 446) in a therapy session, the number of particular successive cycles (e.g., number of successive cycles through box 446 or box 448 or box 452 or box 454 or box 458), the patient characteristics, environmental conditions, procedural approach, and/or product cycle lifetime (i.e., operable age), among other aspects. By deduction, in box 450, the threshold current value has not been achieved in the present cycle, although in some embodiments, affirmative determination and/or confirmation that threshold current value has not been achieved may be performed. Responsive to determination that the maximum duration has not been achieved, control proceeds to box 452. Otherwise, responsive to determination that maximum duration has been achieved, control proceeds to box 454.

In box 452, determination is made to apply the updated duration. In the illustrative embodiment, the duration has been updated in box 448 by increasing the presently selected duration by the predetermined duration interval, and determination to apply the updated duration confirms and proceeds with the updated duration. In the illustrative embodiment, the applied voltage remains as presently selected. Proceeding with the updated duration proceeds openly to return to box 442, to again apply electrical energy to the discharge electrodes to delivery therapy using the updated duration.

In box 454, the applied voltage is increased by a predetermined voltage interval. The presently selected setting for applied voltage is illustratively increased by the predetermined voltage interval. The presently selected duration is returned to the value at the initial setting, exampled as 0.5 microseconds, to be applied with the updated voltage, although in some embodiments, the updated duration may have any suitable value under newly updated voltage settings, for example, the updated duration may be determined based on the number of cycles of the therapy session when newly update voltages occur.

In the illustrative embodiment, the predetermined voltage interval is embodied as a fixed value of 250 V, such that, in the first exemplary occurrence of box 454, the presently selected applied voltage is increased to 2750 V from the value at the initial setting of 2500 V. In some embodiments, the predetermined voltage interval for a given cycle may be determined based on the number of previous cycles in a therapy session, the number of cycles maintaining present settings (e.g., through box 446) in a therapy session, the number of particular successive cycles (e.g., number of successive cycles through box 446 or box 448 or box 452 or box 454 or box 458), the patient characteristics, environmental conditions, procedural approach, and/or product cycle lifetime (i.e., operable age), among other aspects.

In box 456, a determination is made whether the maximum voltage has been achieved. In the illustrative embodiment, the maximum voltage is a predetermined voltage embodied as a fixed value, e.g., 3500 V. By way of example, if the control sequence progressed through cycles from initial settings of box 440 at 2500 V through box 454, until reaching 3500 V, the maximum voltage would be achieved as a threshold value.

In some embodiments, the maximum voltage for a given cycle may be determined based on the number of previous cycles in a therapy session, the number of cycles maintaining present settings (e.g., through box 446) in a therapy session, the number of particular successive cycles (e.g., number of successive cycles through box 446 or box 448 or box 452 or box 454 or box 458), the patient characteristics, environmental conditions, procedural approach, and/or product cycle lifetime (i.e., operable age), among other aspects. By deduction, in box 456, the threshold current value has not been achieved in the present cycle, although in some embodiments, affirmative determination and/or confirmation that threshold current value has not been achieved may be performed. Responsive to determination that maximum voltage has not been achieved, control proceeds to box 458. Otherwise, responsive to determination that maximum voltage has been achieved, control proceeds to box 460.

In box 458, determination is made to apply updated voltage. In the illustrative embodiment, the voltage has been updated in box 454 by increasing the presently selected voltage by the predetermined voltage interval, and determination to apply the updated voltage confirms and proceeds with the updated voltage. The duration was updated in box 445 to return to the value at the initial setting, exampled as 0.5 microseconds, and proceeds to be applied with the updated applied voltage. Proceeding with the updated voltage proceeds openly to return to box 442, to again apply electrical power to the electrodes to delivery therapy using the updated voltage and updated duration.

In box 460, determination of error occurs. Responsive to determination of error, an error message is provided. Such error message illustratively terminates the therapy session, but in some embodiments, may take other safety and/or communication actions, for example, such as displaying an error communication to a user.

In the illustrative embodiment, by deduction, responsive to error determination, the maximum voltage and maximum duration can be determined not to have generated a spark, although in some embodiments, insufficient spark generation may be determined. In some embodiments, responsive to error determination, failed and/or insufficient spark generation under maximum voltage and maximum duration may be determined by affirmative determination and/or confirmation. Following box 460, process control automatically terminate.

Within the discussion of the control 438, exemplary increases in duration have been mentioned, although in some instances, for example, in certain cycles of control 438, voltage level may be decreased, for example, in certain cycles of control 438. For example, duration and/or voltage may be changed in a given cycle according to control 438 to decrease incrementally to achieve appropriate conditions, for example, to achieve appropriate discharged energy as discussed in additional detail herein relative to consideration of the voltage levels of an energy storage system, for example, a capacitance system, 112 before and after discharge.

With reference to FIGS. 6A and 6B, portions of the illustrative electric pulse generation system 120, including portions of the IVL control system 122, are disclosed herein including various features and/or circuitry, although in some embodiments, such systems 120, 122 may share components and/or have isolated components as applicable, for example, such that the circuitry illustrated is intended to be diagrammatic and may also represent circuitry embodied by system 120 alone as applicable. In the illustrative embodiment, the IVL control system 122 includes an adjustable energy storage system, illustratively an exemplary capacitance system, 512 for selective adjustment of the energy storage capacity or magnitude applied for providing electrical energy to the electrodes. We refer hereinafter to energy storage system 512, illustrated by but certainly not limited to, the illustrative capacitance system.

The energy storage system 512 receives charge electrical energy from a power source of the electric pulse generation system 120. The energy storage system 512 provides discharge electrical energy to the electrodes (e.g., illustratively via VCAP2 and grounding), as discussed in additional detail herein.

The adjustable energy storage system 512 illustratively includes a number, e.g., one or more, of energy storage units, exemplified as individual energy storage element 514 (e.g., capacitors), defining an energy storage network. In the illustrative embodiment, each energy storage element 514 may be sized to have the same energy storage capacity and may be arranged for parallel connection with the other energy storage unit(s) in the energy storage network, although in some embodiments, any suitable size and/or arrangement of the energy storage unit(s) may be provided to support variable energy storage for IVL therapy. A relay system 516 may be arranged in connection with at least some of the energy storage elements 514 of the network. The relay system 516 comprises one or more relays for selectively connecting energy storage element(s) 514 together to receive charge and to discharge electrical energy to the electrodes.

In the illustrative embodiment, the relay system 516 includes an engaged arrangement in which all energy storage elements 514 of the network are connected for IVL use. Energy storage element(s) 514, when connected for IVL use, may be connected with other portions of the electric pulse generation system 120 to exchange electrical energy under other control operations. For example, under typical charge control operations, energy storage elements 514 connected for IVL use by relay system 516 may receive charge from power supply, and/or under typical discharge control operation, energy storage elements 514 connected for IVL use by relay system 516 may provide discharge energy to the electrodes 18. Accordingly, it can be appreciated that the relay system 516 can selectively connect all energy storage element(s) 514 for use in the IVL therapy, to provide a maximum energy storage magnitude.

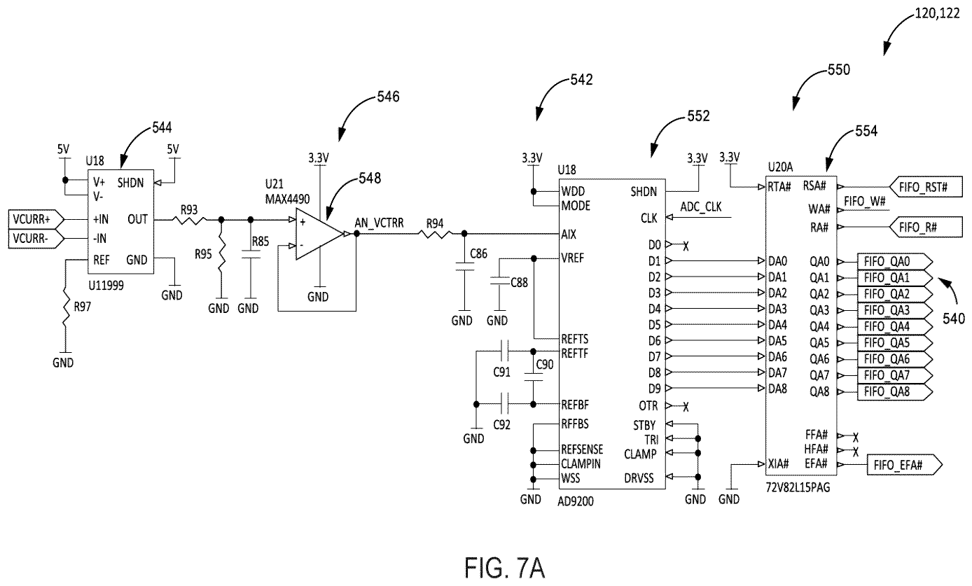

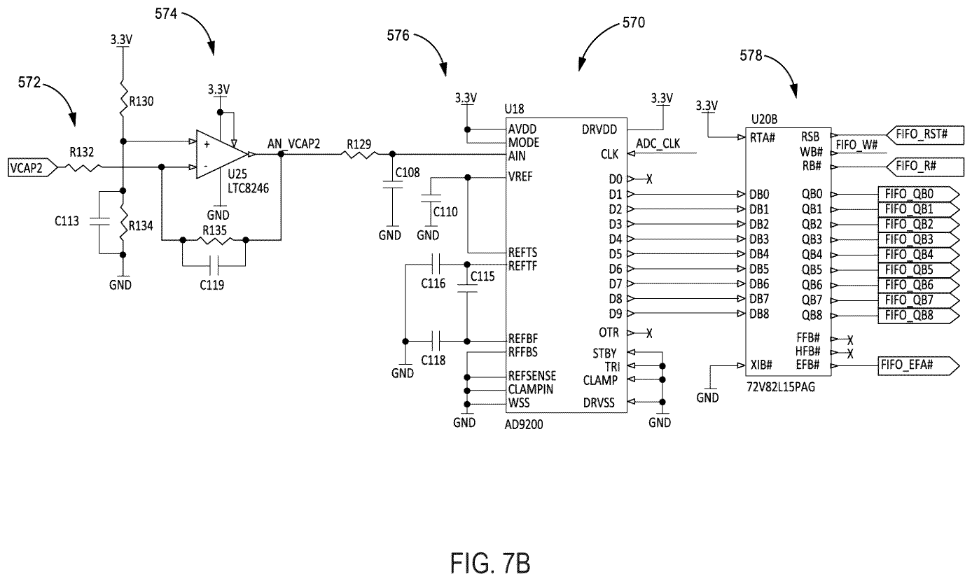

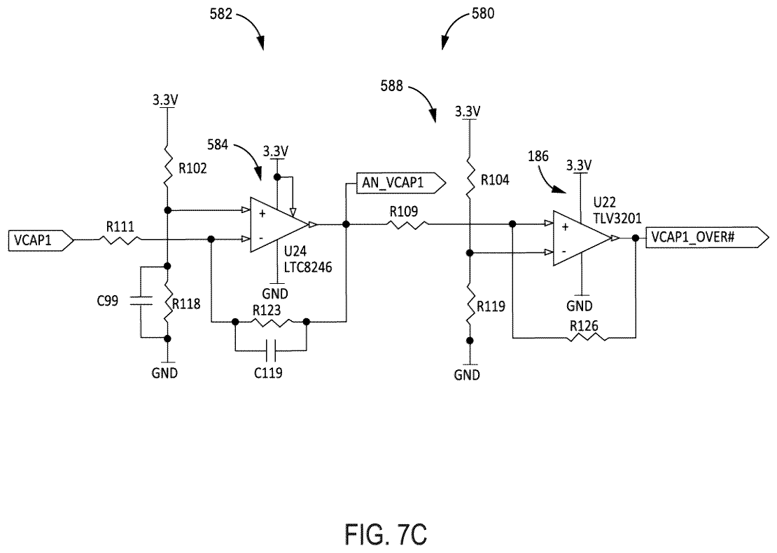

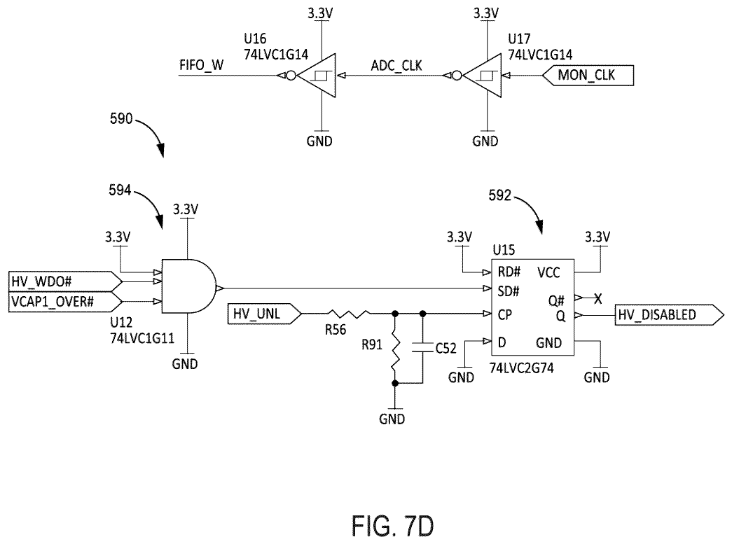

Additionally, the relay system 516 includes a disengaged arrangement in which fewer than all energy storage elements 514 of the network are connected for IVL use as suggested in FIGS. 7A-7D. A number of energy storage elements 514, illustratively two energy storage elements, are rendered disconnected from the other energy storage element(s) by disengagement of the relay system 516 for ease of description but without limitation. Energy storage elements 514 disconnected for IVL use by relay system 516 cannot receive charge from the power supply, and/or under typical discharge control operation, energy storage elements 514 disconnected for IVL use by relay system 516 cannot provide discharge energy to the electrodes 18. Energy storage elements which are disconnected for use in IVL therapy may be discharged apart from the electrodes (e.g., for safe reduction of stored power), illustratively via diode 518 arranged in parallel with the relay system 516.

It can be appreciated that by adjustment of the energy storage capacity available to provide electrical energy to the electrodes, the total amount of energy provided to the electrodes can be controlled. The stored energy can be indicated according to ½C*V², where V represents the voltage and C represents the capacitance, such that applied electrical energy is directly proportional to the amount of energy connected in use. Accordingly, by selectively engaging the relay system 516 to close the circuit and connect the disconnected energy storage element(s), the energy storage capacity of the IVL system can be adjusted to provide variable energy levels to the electrodes. Although the illustrative embodiment includes relay control for connection and/or disconnection of a pair of energy storage elements, any suitable number of relays and/or energy storage elements may be applied to provide adjustable energy storage capacity for IVL therapy.

In the illustrative embodiment, the IVL control system 122 is configured to govern the applied stored energy. The IVL control system 122 illustratively determines the amount of stored energy to be applied, and upon determination that a change in the energy storage magnitude is desired, the IVL control system 122 operates the relay system 516 accordingly. For example, the IVL control system 122 may determine that one voltage pulse desires and/or requires lower energy storage magnitude and may communicate to operate the relay system 516 in the disengaged arrangement.

For one or more subsequent voltage pulses, the IVL control system 122 may determine that another voltage pulse desires and/or requires greater energy storage magnitude and may communicate to operate the relay system 516 in the engaged arrangement. For one or more further subsequent voltage pulses, the IVL control system 122 may determine that lower energy storage magnitude is again desired and/or required and may return the relay system 516 to the disengaged arrangement. Accordingly, the IVL control system 122 may operate the relay system 516 as needed to provide adjustable energy storage magnitudes for any given voltage pulse within a series of voltage pulses.

The applied energy storage may be adjusted on an ongoing basis, for example, for any given pulse. In practice, adjusting the energy storage capacity or magnitude may be conducted in conjunction with the level of voltage to be applied and/or in consideration of other aspects of power, efficiency, and/or technique. Moreover, under the lifetime of applied devices and systems, typical wear to components can alter the electrical and/or physical characteristics thereof, which may benefit from adjustment to the energy storage that is applied. For example, even small wear to electrodes can vary the spacing (gap) between a set of electrodes which can alter the conditions of the arc between electrodes. Accordingly, adjustable energy storage magnitude can accommodate variations in the electrodes and/or or portions of the discharge system under repeated use, whether in individual therapy sessions or otherwise.

With continued reference to FIGS. 6A and 6B, embodiments of the disclosure are directed to systems and methods for adjusting the voltage provided to charge the energy storage system 512 as charge voltage. The charge voltage is illustratively provided as input to the energy storage system 512 as energy accumulation for discharge to generate a voltage pulse, controlled for variable charge. The charge voltage is illustratively controlled by a charge control system 520 of the IVL control system 122.

In the illustrative embodiment, the charge control system 520 provides accurate control of charge voltage via high-frequency switched control signals from the processor 124. The switched control signal (“HVIN_VSET”), illustratively embodied as a pulse width modulated (PWM) signal, is amplified for control of high voltage supply. A high voltage DC/DC converter system 522 receives indication of the switch control signal within the range of about 0 V to about 12 V, and provides a corresponding charge voltage illustratively within the range of about 0 V to about 4000 V.

In the illustrated embodiment, the charge control system 520 includes a buck regulator system 524 for conditioning the low voltage power. The buck regulator system 524 is illustratively embodied as an integrated circuit (IC) to provide signal conditioning with low pass filtering and buffering of the PWM signal. The buck regulator system 524 receives a conditioned PWM signal with feedback for providing controlled low voltage power to the converter system 522 for applying high voltage power.

Resistors 526 can scale down the feedback voltage appropriately for the IC operation, to provide a variable feedback voltage to the buck regulator system 524 within a range of about 0 V to about 3.3 V at the additional resistor 528. As the duty-cycle of the PWM signal increases from zero to 100 percent, the filtered signal increases from zero volts to 3.3 volts, which can increase the current provided to the feedback network, and can require less voltage to achieve regulation, for example, at resistors, inductors, and/or capacitors arranged between the buck regulator system 524 and the converter system 522.

Referring still to FIGS. 6A and 6B, embodiments of the disclosure are directed to systems and methods for controlling the active time for discharge voltage pulses provided to the electrodes. A switching signal (e.g., “GATE_PULSE”) is provided by the processor 124 for high voltage switching via low voltage signaling. In the illustrative embodiment, the switching signal is applied to precisely activate a discharge switch system 530. The discharge switch system 530 is illustratively embodied to implement a driver 531 and semiconductor devices 532 as gate switches.

The gate switches 532 are illustratively embodied as insulated gate bipolar transistors (IGBT) having n-type gate-controlled arrangement. On active state of the switching signal to the driver 531, the gate switches 532 are activated into their conducting state to communicate discharge of energy from the energy storage system 512 to the electrodes. On inactive state of the switching signal the gate switches 532 are deactivated into their non-conducting states, blocking discharge of the energy storage system 512 to the electrodes.

In the illustrative embodiment, the gate switches 532 are arranged as active high devices, although in some embodiments, may be implemented as active low devices. Gate-controlled, active high IGBTs can provide precision control of discharge from the energy storage system 512, but may be implemented by any suitable manner including by other suitable semiconductors (e.g., p-type, FET, etc.) and/or other control designs (e.g., collector, emitter control, etc.).

In the illustrative embodiment, the discharge switch system 530 includes an anti-parallel diode 534 arranged to reduce reverse-voltage stresses on the gate switches 532. A disable signal (“HV_DISABLED”) is provided to the driver 531 which permits discharging of energy to the electrodes on inactive (low) signal, and may activate (high) to disable high voltage discharge under direction of the processor 124 and/or other safety systems. The snubber system 536 is illustratively embodied as a resistor-capacitor-diode (RCD) snubber network arranged to reduce voltage transients that may exceed rated voltages of various high-voltage components.

Referring now to FIGS. 7A-7D, the IVL control system 122 illustratively includes electrical power monitoring system 540. The electrical power monitoring system 540 is configured to conduct monitoring of various parameters of electrical power of the IVL device and systems, illustratively including sensing of current and voltage delivered to the electrodes, and voltage of the adjustable energy storage system 512.

The electrical power monitoring system 540 illustratively includes a current monitoring system 542. The current monitoring system 542 is embodied to sense the current delivered to the electrodes for a given voltage pulse. As discussed in additional detail herein, the current delivered to the electrodes can be considered in determining the power characteristics for subsequent voltage pulses.

In the illustrative embodiment as shown in FIGS. 4A-4D, the current monitoring system 542 receives indication of the voltage levels applied in each voltage pulse for determining current delivered to the electrodes. Returning briefly to FIGS. 3A and 3B, a shunt resistor 538 is arranged within the high-voltage current path establishing a proportional voltage (e.g., “VCURR+”, “VCURR−”). The proportional voltage is communicated to the current monitoring system 542 as shown in FIGS. 4A-4D.

A chip comprising an amplifier 544 is arranged to scale-up the proportional voltage, and provides the analog result to a conditioning network 546, embodied to include a resistor-capacitor network for scaling and/or filtering. The conditioning signal is buffered by a buffering amplifier 548, the output of which is provided to an analog-to-digital conversion (ADC) system 550 for digital conversion.

The ADC system 550 illustratively includes a converter 552 and memory 554. In the illustrative embodiment, the converter 552 provides digital output from the analog input, and the memory 554 is embodied as a first-in-first-out (FIFO) device for intermediate storage of digital outputs. The memory 554 illustratively receives the same clock signal driving the converter 552, to allow quick sampling of a number of measurement points with low-jitter. Memory outputs are provided to the processor 124 for consideration in overall IVL therapy control.

The IVL control system 122 illustratively includes a current monitoring system which compares the output signal (“VCURR”) generated by the chip comprising an amplifier 544 with a threshold value. The threshold value is embodied to be generated by a variable duty cycle PWM signal (“ISNS_ISET”) from the processor 24. The PWM signal can be low-pass filtered and/or buffered before delivery to a comparator. Responsive to the measured current exceeding the threshold value, the current monitoring system can assert an error signal (e.g., “ISNS OVER #”) to avoid overcurrent conditions.

The electrical power monitoring system 540 illustratively includes a voltage monitoring system 570. The voltage monitoring system 570 is embodied to sense the voltage between the set of electrodes. As discussed in additional detail herein, the voltage between the electrodes for a given pulse can be considered in determining the power characteristics for subsequent voltage pulses.

In the illustrative embodiment, the voltage monitoring system 570 includes a resistor network 572 arranged to attenuate the (switched) voltage of one of the electrodes of a set (e.g., “VCAP1”). The attenuated signal is provided to an op amp network 574 for filtering and offsetting for output to digital conversion. The output from the op amp network 574 is provided to the ADC conversion system 576 for digitization, including storage in FIFO memory 578 for access by the processor 124.

The electrical power monitoring system 540 may illustratively include an energy storage capacity voltage monitoring system 580 configured for monitoring the voltage within the adjustable energy storage system 512. Monitoring the voltage of the energy storage system 512 can allow determination of the stored energy of the energy storage system 512. Moreover, comparison of the stored energy of the energy storage system 512 before and after discharge can provide indication of the total energy delivered during a given discharge cycle. Such total energy data can be considered to increase confidence in determining whether a sufficient spark has been generated for IVL therapy.

In the illustrative embodiment, the voltage monitoring system 580 includes a voltage limiting system configured to monitor net voltage of the energy storage system 512 during charging. In the illustrative embodiment, voltage monitoring is discussed relative to the connected energy storage elements 514, more specifically, those energy storage elements which are connected to provide controlled discharge energy for IVL therapy, and not energy storage elements which are disconnected via the relay system 516 if any.

The voltage monitoring system 580 receives indication of the voltage of the energy storage system 512 during charging (“VCAP1”). The system 580 illustratively includes an amplifier configuration 582 comprising an amplifier 584 and a comparator 586. The comparator 586 is illustratively arranged to compare to the voltage with a fixed voltage, and to responsively trigger a signal (e.g., “VCAP1_OVER #”) when the voltage of the energy storage system 512 exceeds the fixed voltage. In the illustrative embodiment, the fixed voltage is embodied as setpoint generated by a resistor-resistor-capacitor network 588 above normal operation, but before damage will occur to various HV components.

An intermediate voltage of this circuit (e.g., “AN_VCAP1”) can be used to monitor progress during the charging cycle of the energy storage system 512. The intermediate voltage illustrative represents a heavily attenuated indication of the high voltage provided by the energy storage system to the electrodes (“VCAP_1”). Such attenuated signal can allow monitoring of high voltage systems while handling lower voltage indications thereof.

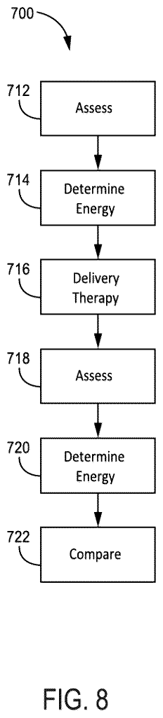

Referring now to FIG. 8, and with continued reference to FIGS. 1 and 3, the IVL system 12 can consider energy of the energy storage system 512 in operation. By monitoring energy of the energy storage system 512 before and after a discharge event, indication of the generation (and/or sufficiency) of spark can be determined as discussed in additional detail regarding the illustrative embodiment with reference to operation 700 concerning boxes 712 through 722.

In box 712, assessment of the energy storage system 512 is conducted. In the illustrative embodiment, the assessment includes determination of a voltage of the energy stored by the energy storage system 512. As mentioned above, the voltage monitoring system 580 can monitor voltage of the energy storage system 512, for example, via the voltage limiting system during charging. In some embodiments, the assessment may include determination of any other suitable parameters to support energy monitoring of the energy storage system 512.

In box 714, the energy of the energy storage system 512 is determined. In the illustrative embodiment, the energy of the energy storage system 512 is determined based on the measured voltage according to

$$\frac{1}{2} C * {V}^{2} .$$

Accordingly, the processor 124 can compute the current energy of the energy storage system 512, including the energy stored just before discharge of energy to the electrodes.

In box 716, IVL therapy can be attempted. In the illustrative embodiment, a voltage pulse can be delivered to the electrodes. The voltage pulse can be applied according to the control arrangement as mentioned herein, for example, based on a determined duration in a control sequence.

In box 718, assessment of the energy storage system 512 is conducted. Assessment of the energy storage system 512 in box 718 is embodied as occurring immediately after attempted IVL therapy in box 716 to provide an indication of the energy state of the energy storage system 512 immediately after (attempted) discharge to the electrodes. In the illustrative embodiment, the assessment includes determination of a voltage of the energy stored by the energy storage system 512, embodied as conducted by the voltage monitoring as mentioned above, although in some embodiments, assessment of the energy storage system 512 in box 718 may differ from box 712 in methodology and/or practice.

In box 720, the energy of the energy storage system 512 is determined. In the illustrative embodiment, the energy of the energy storage system 512 is again determined based on the measured voltage according to

$$\frac{1}{2} C * {V}^{2}$$

just as in box 714, yet after attempted delivery of IVL therapy. In some embodiments, determining energy of the energy storage system 512 in box 720 may differ in methodology and/or practice from that in box 714. Accordingly, the processor 124 can compute the current energy of the energy storage system 512, including immediately after (attempted) discharge of energy to the electrodes.

In box 722, comparison between energy determinations is conducted. The amount of energy determined within the energy storage system 512 in box 714 is illustratively subtracted from the amount of energy determined within the energy storage system 512 in box 720, such that the result represents the amount of energy discharged from the energy storage system 512 under a single attempt to deliver IVL therapy.

The amount of energy discharged can be considered to determine whether a spark (or sufficient spark) has occurred such that IVL therapy occurs. In the illustrative embodiment, a threshold energy discharge represents a discharged energy level which confidently indicates spark sufficient for IVL has occurred. Accordingly, in box 722, comparing the stored energy levels before and after discharge to determine whether the threshold energy discharge has been achieved can indicate spark for IVL therapy.

In the illustrative embodiment, and with reference to FIG. 5, the threshold energy discharge is a fixed predetermined value, for example, 600 millijoules (e.g., 3700 V, 90 nanofarad). However, in some embodiments, the threshold energy level for a given cycle may be determined based on the number of previous cycles in a therapy session, the number of cycles maintaining present settings (e.g., through box 446 of FIG. 5) in a therapy session, the number of particular successive cycles (e.g., number of successive cycles through box 446 or box 448 or box 452 or box 454 or box 458), the patient characteristics, environmental conditions (e.g., location within patient's body such as above the knee or above the knee), procedural approach, and/or product cycle lifetime (i.e., operable age), among other aspects.

Comparison of energy levels of the energy storage system 512 which indicates a spark for IVL therapy has occurred can responsively cause further therapy at the same duration, energy level, threshold characteristic, and/or threshold adjusted for other parameters. Comparison of energy levels of the energy storage system 512 which indicates a spark for IVL therapy has not occurred can cause adjustment to the duration and/or energy level applied, for example, as discussed concerning control operation 38.

In some embodiments, the threshold current value can be applied together with the threshold energy discharge, such that either threshold can individually indicate a spark for IVL therapy. In some embodiments, both thresholds may be required to be met to indicate a spark for IVL therapy.

Consideration of the energy states of the energy storage system 512 can provide desirable monitoring of IVL operations. For example, such monitoring can be less intrusive by reducing the need for direct measurements at the electrodes. Moreover, in high power applications, reliable consideration of the energy states can promote confidence over mere direct measurement in unpredictable high energy arc scenarios.

The IVL control system 122 illustratively includes an external watchdog system configured to assist in safe operation. The watchdog system includes an integrated circuit configured to trigger an error under lack of a timely toggled input signal to ensure appropriate high voltage operation. In some embodiments, the watchdog system may be formed externally including processor, memory, and/or circuitry distinct from or shared with the IVL control system 122.

Returning to FIGS. 7A-7D, in the illustrative embodiment, the IVL control system 122 includes an umbrella monitoring system 590 configured to assist in safe operation. The umbrella monitoring system 590 illustratively includes a flip flop 592 and logic gate 594 for consideration of monitoring signals. The logic gate 594 is arranged to receive monitoring signals, embodied as energy storage system overvoltage (“VCAP1_OVER #”) from the voltage monitoring system 580, high voltage warning (“HV_WDO #) from the watchdog system, and in some embodiments, may receive overcurrent from the current monitoring system (“ISNS_OVER #”).

The logic gate 594 is embodied as an AND gate and the flip flop 592 embodied as an asynchronous D-flip flop, such that activation signals from the gate 594 which last longer than the minimum clock pulse width of the flip flop 592 cause an assertion of outputs to disable high voltage output (e.g., “HV_DISABLED”), but activation signals from the gate 594 shorter than the minimum clock pulse width of the flip flop 592 do not raise disabling outputs from the umbrella monitoring system 590.

Assertion of the signal to disable high voltage output (“HV_DISABLED”) is illustratively provided to the discharge switch system 530 to disable voltage pulse switch activation to the electrodes. In the illustrative embodiment, the disabling output signal is provided to the driver 531 and indirectly to alter on/off operation of the gate switches 532. Such disabling output signal is illustratively provided to low-voltage supplies, e.g., buck regulator system 524, and high voltage modules, e.g., converter system 522.

Accordingly, the logic gate 594 receives monitoring signals discussed above comprising: (1) energy storage system overvoltage (“VCAP1_OVER #”) from the voltage monitoring system 580, (2) high voltage warning (“HV_WDO #) from the watchdog system, and in some embodiments, (3) may receive overcurrent from the current monitoring system (“ISNS_OVER #”). These monitoring signals ae also connected to a three-input AND logic gate [U12], which is upstream of a D flip-flop with asynchronous set and reset functionality [U15], so that any signal that asserts for longer than the minimum pulse width of the flip-flop [U15] will cause its outputs [HV_DISABLED, HV_DISABLED #] to assert. These signals travel downstream and inhibits the operation of the gate driver [U11], variable low-voltage supply [U6], high-voltage module [U7], and slightly changes the turn-on and turn-off operation of the switching devices [Q10, Q11] via transistors [Q12, Q13].” This system allows any one of monitoring signals to disable the output of the system if asserted longer than an established duration.

Within the present disclosure, the ability to run off of AC mains or battery DC can afford versatility of power and control for IVL therapy. Unlike known IVL systems, certain embodiments within the present disclosure can avoid sitting idle until fully (or substantially) recharged in order to be applied in IVL therapy, for example, if insufficiently charged by the time IVL therapy is desired. Accordingly, such costly delays, or interruptions, in procedure can be avoided with embodiments of the present disclosure. The electric pulse generation system 120 illustratively includes a battery power storage system, and is configured to selectively charge the energy storage system 512 from battery stored energy only, from the battery power storage system while connected to mains, such as outlet power, or directly from DC power converted from the AC mains without the battery power storage system. When plugged in to AC mains, regulated DC power is delivered directly to the high voltage systems. In operational states in which the current demand for IVL operations is high, the battery power storage system charge current can be reduced to allow for higher IVL operations system current. When not plugged in to AC mains, battery power can be delivered directly to energy storage system 512. In the illustrative embodiment, systems and devices of power management, including for example, inverters, conditioners, power storage devices, and/or related aspects may be comprised by the electric pulse generation system 120 to provide applicable power to the IVL control system 122.

Within the present disclosure, consideration of a set of discharge electrodes has been discussed in the context of a pair of electrodes, one of which may serve as a cathode and the other of which may serve as an anode, at a given instance. However, the number of electrodes in a set may be greater than a pair, for example, including one or more cathodes communicating with one or more anodes. Additionally, devices, systems, and methods within the present disclosure may include more than one communicating group of electrodes, whether electrically arranged serially, in parallel, or independently from each other.

Power control operations disclosed herein may be applied equally, simultaneously, and/or sequentially to individual sets or groups of electrodes, in a given IVL therapy cycle. For example, threshold current value may be applied collectively to all deployed electrodes or to individual groups or sets of electrodes. Determinations made with respect to power control may be applied equally to related electrodes, or may be individualized to groups or sets of electrodes, in a given IVL therapy cycle. Within the present disclosure, supporting components, such as power supplies, sensors, and other implementing structures and/or features for performing IVL operations as disclosed herein are embodied as sub-portions of the electric pulse generation system 120 and/or IVL control system 122, for example, as parts of circuitry and/or instructions.

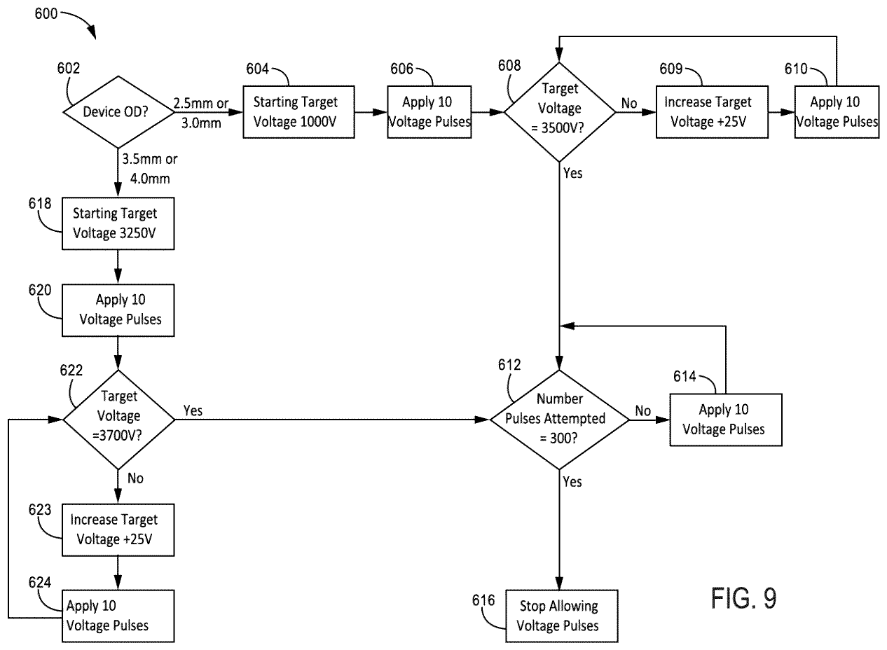

Referring now to FIG. 9, an exemplary flow diagram is shown concerning another embodiment of a control 600 in operation of an embodiment of an IVL system, specifically the number of voltage pulses generated and the magnitude of the generated pulses. It is understood that the control 600 may be combined with aspects of the control system and method embodiments described above.

Control operations of control 600 may be governed by the electric pulse generation system 120, and illustratively by the IVL control system 122 discussed above. As discussed further herein, control system 122 may control the number of generated voltage pulses in a series (or a plurality of series) of voltage pulses. An acceptable voltage window comprises a predetermined starting voltage magnitude and a predetermined upper voltage magnitude. IVL control system further comprises a predetermined voltage magnitude for incremental increasing of voltage magnitude after each series of voltage pulses if the magnitudes of the executed voltage pulses is within the acceptable voltage window. Separate sets of predetermined control data may be provided in control system 122 for IVL systems comprising balloons that are of identifiable characteristics such as, without limitation, different sizing, e.g, 2.5 mm, 3.0 mm, 3.5 mm and/or 4.0 mm.

An exemplary embodiment of an IVL system may comprise a 2.5 mm or a 3.0 mm balloon, wherein the control system 122 comprises control data comprises an exemplary starting target voltage of 3000V (predetermined lower voltage threshold), voltage pulse series comprising an exemplary 10 pulses, and an exemplary incremental voltage increase of 25V if the voltage pulse series magnitudes are less than an exemplary upper voltage threshold of 3500V. As the skilled artisan will recognize, the incremental voltage increase may comprise any voltage magnitude, including without limitation within 1V to 250V. An exemplary voltage increase may comprise 25V, but may be greater or less than 25V in certain embodiments. As the artisan will recognize, the predetermined starting voltage may be less than 3000V and the predetermined upper voltage threshold may be greater than 3500V. Thus, an exemplary starting voltage may, without limitation, comprise 2500V and an exemplary upper voltage threshold may comprise 4100V. In other embodiments, an exemplary predetermined starting voltage may be greater than 3000V and an exemplary upper voltage threshold may be greater than 3250V.

Another exemplary embodiment may comprise a 3.5 mm or a 4.0 mm balloon, wherein the control system 122 comprises control data comprises an exemplary starting target voltage of 3250V (predetermined lower voltage threshold), voltage pulse series comprising 10 pulses, and an incremental voltage increase of 25V if the voltage pulse series magnitudes are less than an exemplary predetermined upper voltage threshold of 3700V.

FIG. 9 illustrates initiation of a voltage pulse generation and control system that begins with box 602 which requires determination of a particular balloon characteristic of interest, for example the outer diameter (“OD”) of the IVL system's balloon. In a first embodiment, if the balloon's outer diameter is, e.g., 2.5 mm or 3.5 mm, then in box 604 the starting voltage is set to 3000V, also referred to as the predetermined lower voltage threshold of the acceptable voltage magnitude window). The IVL therapy is initiated in box 606 by application of a series of voltage pulses (or shocks) from the electric pulse generation system 120 wherein each voltage pulse travels to the electrodes 18 within the balloon 16. If, in box 608, the target voltage magnitude is not at the predetermined upper voltage threshold, e.g., 3500V, then as in box 609, the target voltage is increased by an exemplary 25V (from 3000V to 3025V) and another series of voltage pulses (in this case 10 pulses) is executed, as in box 610 at 3025V. This process continues with cycling between boxes 608, 609 and 610 until the target voltage is at 3500V. When the target threshold voltage, or predetermined upper voltage threshold, is reached, and/or in some embodiments a predetermined maximum or desired number of voltage pulses, e.g, 300 pulses (or shocks) have been generated, then as in box 612, control system 122 determines if the number of generated voltage pulses (or shocks) in the plurality of series of voltage pulses has reached a maximum, or desired, number of pulses, e.g., 300 voltage pulses. If the maximum or desired, e.g., 300, voltage pulse threshold has not been reached, then as in box 614 another series of, e.g., 10, voltage pulses (or shocks) are applied. When the maximum or desired, e.g., 300 voltage pulse threshold has been reached, then as in box 616, additional voltage pulses (or shocks) are not allowed.

The predetermined maximum number of voltage pulses in various embodiments of the present disclosure may be within the range of 10-300 voltage pulses. The exemplary embodiments discussed herein comprise a predetermined maximum number of voltage pulses equal to 300 pulses. In other embodiments, the maximum number of voltage pulses may be greater than 300 pulses.

In a second embodiment, if the balloon's outer diameter is determined in box 602 to be, e.g., 3.5 mm or 4.0 mm, then the electric pulse generation system 120 initiates therapy at box 618 the starting voltage is set to 3250V, also referred to as the predetermined lower voltage threshold of the acceptable voltage magnitude window). The IVL therapy is initiated in box 620 by application of a series of voltage pulses from the electric pulse generation system 120 wherein each voltage pulse travels to the electrodes 118 within the fluid filled member 116. If, in box 622, the target voltage magnitude is not at the predetermined upper voltage threshold, e.g., 3500V, then as in box 623, the target voltage is increased by an exemplary 25V (from 3250V to 3275V) and another series of voltage pulses (in this case 10 pulses) is executed, as in box 624, at 3275V. This process continues with cycling between boxes 622, 623 and 624 until the target voltage is at 3700V. When the target threshold voltage, or predetermined upper voltage threshold, is reached, and/or in some embodiments a maximum or desired number of pulses, e.g, 300 pulses (or shocks when applied to the one or more pairs of spaced-apart electrodes) have been generated, then as in box 612, control system 122 determines if the number of generated voltage pulses (or shocks) in the plurality of series of voltage pulses has reached a maximum, or desired, number of pulses, e.g., 300 voltage pulses. If the maximum or desired, e.g., 300, voltage pulse threshold has not been reached, then as in box 614 another series of, e.g., 10, voltage pulses (or shocks) are applied. When the maximum or desired, e.g., 300 voltage pulse threshold has been reached, then as in box 616, additional voltage pulses (or shocks) are not allowed.

Alternatively, the physician conducting the IVL therapy according to the voltage pulse generation and control system may determine that the therapy is complete at a point during execution of the therapy. If the therapy is determined to be completed, then the physician may terminate the process of the voltage pulse generation and control system at any point.

In some embodiments, the voltage pulse generation and control system may comprise modification of the duration of applied voltage within, or across, one or more of the plurality of series of voltage pulses in accordance with the embodiments discussed above in connection with FIG. 5. For example, duration may be increased, or decreased, by a predetermined duration interval, illustratively embodied as a fixed value, e.g., 0.5 microseconds. In some embodiments, the predetermined interval for given cycle may be determined based on factors such as the number of times therapy cycles have ensued previously during the therapy session (e.g., number of series of voltage pulses that have been executed by proceeding through boxes 606, 608 and 610, or 620, 622 and 624), the patient characteristics, environmental conditions, procedural approach, and/or product cycle lifetime (i.e., operable age), among other aspects. In some embodiments, the predetermined duration interval for a given cycle may be varied by predetermined rate of change, for example, by percentage gain or loss by cycle.

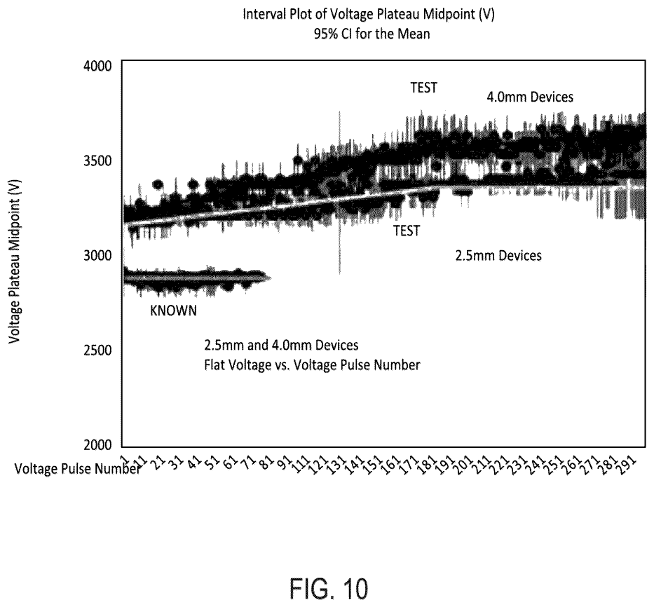

FIG. 10 presents a graphic comparison of known IVL devices comprising 2.5 mm and 4.0 mm OD balloons (KNOWN) with IVL devices according to the present disclosure and comprising 2.5 mm and 4.0 mm OD balloons (TEST).

The KNOWN devices permit 80 voltage pulses or shocks to be generated. The TEST devices generated 300 voltage pulses or shocks. During the comparative testing, for each voltage pulse, the voltage peak magnitude was obtained and plotted for each tested device. The KNOWN devices provide a relatively flat or constant voltage for each voltage pulse or shock number and begin at a lower voltage magnitude than the TEST devices. The TEST devices were operated and controlled in accordance with the disclosed embodiments herein.

In contrast, each of the 2.5 mm and 4.0 mm TEST devices begin at a higher voltage magnitude than the KNOWN devices. The 2.5 mm TEST device begins at a lower voltage than does the 4.0 mm device. As illustrated, both the 2.5 mm and the 4.0 mm TEST device voltage (lower data cluster) rise slowly over the generated voltage pulses, plateauing at approximately 180 pulses, thereafter remaining substantially flat or constant. Referring back to FIG. 9, this pattern of increasing voltage, followed by a flattened or constant voltage region conforms with boxes 604-610 (2.5 mm) and boxes 618-624 (4.0 mm). In each case, the average voltage of the 4.0 mm TEST device is greater than that of the 2.5 mm TEST device.

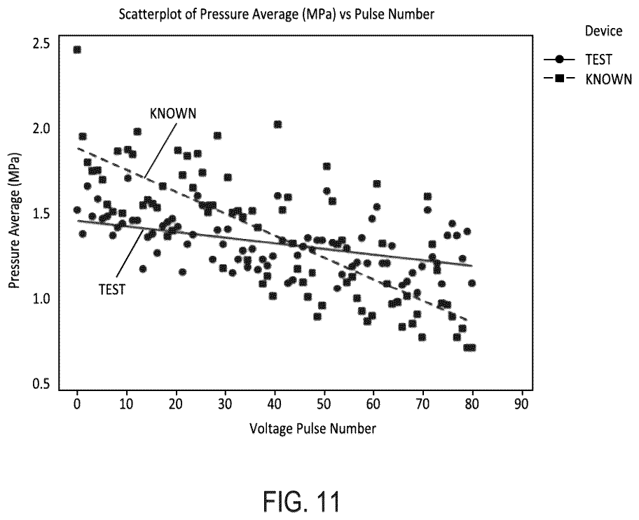

FIG. 11 illustrates a comparison of the TEST and KNOWN IVL devices and a subset of the data of FIG. 10, i.e., comparing the first 80 voltage pulses for each device. Here, the voltage pulse (or shock number) is compared with the average pressure generated during each voltage pulse. The KNOWN data (with a constant voltage in each voltage pulse presents (dashed line) a relatively severe decrease in pressure output as the voltage pulses progress over time. In contrast, the TEST data (solid line) obtained using the voltage and pulse generation algorithm of FIG. 9, presents a pressure output line that decreases at a much smaller angle or slope. Thus, the pressure output of the TEST IVL devices provides a more stable or constant pressure output than the KNOWN IVL devices. The KNOWN IVL devices have a pronounced pressure output decay as the voltage pulses progress. More specifically, the TEST IVL devices provide a decrease in pressure output over 80 pulses that is less than 0.25 MPa.

The pressure output, as tested, was measured in-vitro using pressure sensors (hydrophones) positioned externally to the catheter balloon within the acoustic field generated by device pulse delivery. The device under test and hydrophones were immersed in de-gassed, deionized water maintained at approximately body temperature.

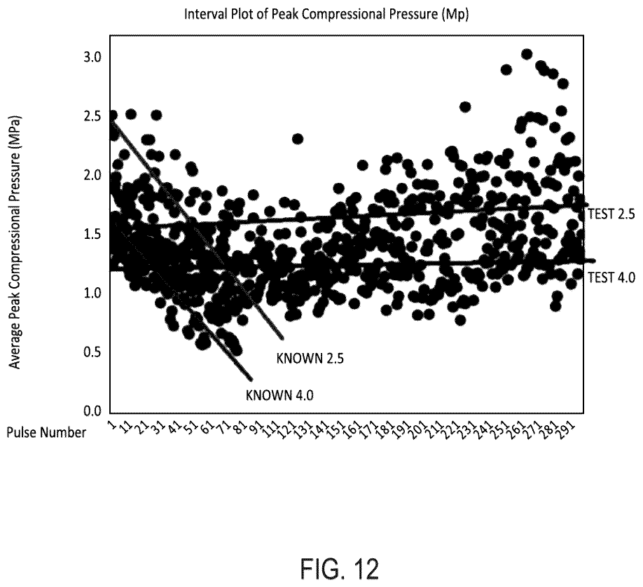

This concept is further demonstrated in FIG. 12, where the average pressure generated by 80 pulses of the KNOWN IVL devices (2.5 mm and 4.0 mm) with constant voltage magnitude is compared with the average pressure generated by the TEST IVL devices (2.5 mm and 4.0 mm) according to the voltage pulse and control method of FIG. 9.

As shown in FIG. 12, the KNOWN 2.5 mm and 4.0 mm devices both provide severely decreasing pressure output slope lines as the voltage pulses progress to 80 pulses. In contrast, the TEST 2.5 mm and 4.0 mm devices provide relatively flat, constant or stable pressure output slope lines as the voltage pulses progress to 300 pulses. Moreover, the slopes of the TEST pressure output lines appear to increase slightly as the voltage pulses progress which may be beneficial in cracking difficult calcified regions. Again, the KNOWN IVL devices have a pronounced and significant pressure output decay over 80 pulses. The TEST IVL devices do not have a pressure decay over 300 voltage pulses.

In summary, the IVL devices operated and controlled according to the present disclosure provide an increasing voltage to the voltage pulses until the upper voltage magnitude threshold is reached. Then, the voltage progress at the upper voltage magnitude threshold until 300 pulses have been executed, or the physical determines therapy is complete. This, as shown above, leads to constant and/or slightly increasing pressure output from each voltage pulse. The pressure output magnitudes, and associated slopes, may be manipulated by modifying the magnitude of each incremental increase in voltage. In some embodiments, the voltage magnitude may be incrementally increased as in FIG. 5. In others, the voltage magnitude may be increased incrementally for at least two series of voltage pulses, then held constant for one or more voltage pulses, then subsequent voltage pulse series may resume the incremental increase in magnitude. In other embodiments, the voltage magnitude may be decreased for one or more series of voltage pulses. All combinations of voltage increase, voltage decrease, and/or no change in voltage over a plurality of a series of voltage pulses in order to manipulate the resulting pressure output are within the scope of the present invention.

In addition, with reference to the above disclosure, various embodiments of the disclosure may provide a pressure output that is substantially the same for all balloon sizes, wherein the balloon sizes may be within a range from 2 mm to 4 mm outer diameter. In these embodiments, larger balloon sizes do not necessarily result in a lower pressure output than the pressure output of a relatively smaller balloon size.

The data of FIGS. 11 and 12 also demonstrate that IVL devices operated according to the present disclosure are also capable of stable operation and pressure output over at least 300 voltage pulses. This is in contrast to the pronounced pressure decay of the KNOWN IVL devices over just 80 voltage pulses.

Moreover, the data of FIGS. 11 and 12 confirm that the IVL control system 122 shown in FIG. 1 is controllable such that the pressure output following an electrical arcing event between two spaced-apart electrodes can be controlled within upper and lower pressure magnitude thresholds or a pressure magnitude window. Further, the pressure output can be controlled using embodiments of the present disclosure in a pattern of increasing pressure over the procedure, decreasing pressure over the procedure, constant pressure over the procedure, and any combination thereof.

Examples of suitable processors may include one or more microprocessors, integrated circuits, system-on-a-chips (SoC), among others. Examples of suitable memory, may include one or more primary storage and/or non-primary storage (e.g., secondary, tertiary, etc. storage); permanent, semi-permanent, and/or temporary storage; and/or memory storage devices including but not limited to hard drives (e.g., magnetic, solid state), optical discs (e.g., CD-ROM, DVD-ROM), RAM (e.g., DRAM, SRAM, DRDRAM), ROM (e.g., PROM, EPROM, EEPROM, Flash EEPROM), volatile, and/or non-volatile memory; among others. Communication circuitry 58 includes components for facilitating processor operations, for example, suitable components may include transmitters, receivers, modulators, demodulators, filters, modems, analog/digital (AD or DA) converters, diodes, switches, operational amplifiers, and/or integrated circuits. In some embodiments, memory 26 may represent one or more memory devices operable for IVL therapy operation. For example, each memory (e.g., 554, 578) may be included as part of memory 26, shared, or isolated therefrom.

Within the present disclosure, consideration of a set of discharge electrodes has been discussed in the context of a pair of electrodes, one of which may serve as a cathode and the other of which may serve as an anode, at a given instance. However, the number of electrodes in a set may be greater than a pair, for example, including one or more cathodes communicating with one or more anodes. Additionally, devices, systems, and methods within the present disclosure may include more than one communicating group of electrodes, whether electrically arranged serially, parallel, or independently from each other.

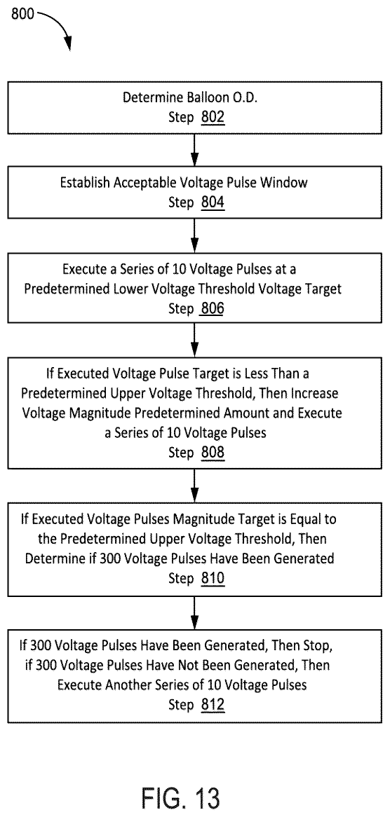

FIG. 13 provides an exemplary flowchart illustrating an exemplary method 800 of one embodiment of the present invention. Thus, step 802 provides for determination of the subject IVL device's balloon outer diameter or OD. This may be done with manual entry into the IVL control system discussed above. Alternatively, connecting the catheter with the IVL control system may provide automatic detection and determination of the balloon's OD. Step 804 provides for establishing an acceptable voltage pulse window, comprising as described above, predetermined lower and upper voltage magnitude thresholds which may be stored in the IVL control system. Step 806 provides for execution of a series of voltage pulses to be controlled and generated by the IVL control system, in an illustrative and exemplary case 10 pulses may be used, at the predetermined lower voltage magnitude threshold. Step 808 provides that if the IVL control system determines that the last executed series of voltage pulses was not executed at the predetermined upper voltage threshold target, then the IVL control system may instruct execution and generation of another series of voltage pulses. Step 810 provides that if the IVL control system determines that the last executed series of voltage pulses was executed at the predetermined upper voltage threshold target, then the IVL control system seeks to determine if the illustrative and exemplary 300 voltage pulses have been executed during the current therapy. If, according to step 812, 300 voltage pulses are determined to have been executed, the IVL control system stops the procedure, allowing no further voltage pulse generation. On the other hand, if 300 voltage pulses have not been executed, then the IVL control system instructs another series of voltage pulses to be executed and at the predetermined upper voltage threshold target magnitude.

In certain embodiments, the devices, systems and methods described herein may comprise 1 pulse/second, 2 pulses/second or 3 pulses/second. In some embodiments, the pulses/second generated by the described embodiments may be within the range of 1 to 5 pulses/second.

Embodiments of the present invention may comprise or utilize a special purpose or general-purpose computer including computer hardware, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: physical computer-readable storage media and transmission computer-readable media.

Physical computer-readable storage media includes RAM, ROM, EEPROM, CD-ROM or other optical disk storage (such as CDs, DVDs, etc.), magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

The functionality described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc.

The present invention may be embodied in other specific forms without departing from its characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

### EXEMPLARY EMBODIMENTS

The following non-limiting and exemplary embodiments are supported by the present disclosure.

### Exemplary Embodiment Set 1

1. An intravascular lithotripsy system comprising:
at least one set of electrodes for arrangement within a body lumen while disposed within an inflatable balloon;
an electric pulse generation system for providing electrical energy to the at least one set of electrode to generate spark for intravascular lithotripsy (IVL) therapy, the electric pulse generation system including an IVL control system comprising a processor for executing instructions stored on a memory, and circuitry configured for communication of signals based on operation of the processor, the IVL control system configured to:
generate an initial series of voltage pulses to the at least one set of electrodes, wherein the magnitude of each voltage pulse in the initial series of voltage pulses comprises a target voltage that is set at a predetermined lower voltage magnitude threshold,
determine whether a threshold parameter is achieved, and
responsive to determination that the threshold parameter is not achieved, increase the voltage magnitude by a predetermined amount, and
generate another series of voltage pulses at the increased voltage magnitude for application to the at least one set of electrodes.

2. The intravascular lithotripsy system of embodiment 1, wherein the IVL control system is configured to continue to determine whether a threshold parameter is achieved after each generated series of voltage pulses.

3. The intravascular lithotripsy system of embodiment 2, wherein the threshold parameter comprises a predetermined upper voltage magnitude threshold.

4. The intravascular lithotripsy system of embodiment 3, wherein the IVL control system is configured to determine if the number of voltage pulses generated does not exceed a predetermined maximum number of voltage pulses.

5. The intravascular lithotripsy system of embodiment 3, wherein the predetermined maximum number of voltage pulses is within a range of 10 to 300 voltage pulses.

6. The intravascular lithotripsy system of embodiment 4, wherein if the IVL control system is configured to determine that the predetermined number of voltage pulses have not been generated, and if the predetermined number of voltage pulses have not been generated, then the IVL control system is further configured to execute another series of voltage pulses at the predetermined upper voltage magnitude threshold.

7. The intravascular lithotripsy system of embodiment 4, wherein if the IVL control system determines that the predetermined number of voltage pulses have been generated, then no further voltage pulses are executed.

8. The intravascular lithotripsy system of any one of embodiments 1-7, wherein the IVL control system is configured to define an acceptable voltage magnitude window comprising the predetermined lower voltage magnitude threshold and the predetermined upper voltage magnitude threshold.

9. The intravascular lithotripsy system, wherein the acceptable voltage magnitude window is different for balloons of different outer diameters.

10. The intravascular lithotripsy system of embodiment 9, wherein the predetermined lower voltage magnitude threshold is about 2500V for balloons having an outer diameter of 2.5 mm.

11. The intravascular lithotripsy system of embodiment 9, wherein the predetermined lower voltage magnitude threshold comprises about 2500V for balloons having an outer diameter of 3.0 mm.

12. The intravascular lithotripsy system of embodiment 9, wherein the predetermined lower voltage magnitude threshold comprises about 2500V for balloons having an outer diameter of 3.5 mm.

13. The intravascular lithotripsy system of embodiment 9, wherein the predetermined lower voltage magnitude threshold comprises about 2500V for balloons having an outer diameter of 4.0 mm.

14. The intravascular lithotripsy system of embodiment 9, wherein the predetermined lower voltage magnitude threshold is about 3000V for balloons having an outer diameter of 2.5 mm.

15. The intravascular lithotripsy system of embodiment 9, wherein the predetermined lower voltage magnitude threshold comprises about 3000V for balloons having an outer diameter of 3.0 mm.

16. The intravascular lithotripsy system of embodiment 9, wherein the predetermined lower voltage magnitude threshold comprises about 3000V for balloons having an outer diameter of 3.5 mm.

17. The intravascular lithotripsy system of embodiment 9, wherein the predetermined lower voltage magnitude threshold comprises about 3000V for balloons having an outer diameter of 4.0 mm.

18. The intravascular lithotripsy system of embodiment 9, wherein the predetermined lower voltage magnitude threshold is less than about 3000V for balloons having an outer diameter of 2.0 mm.

19. The intravascular lithotripsy system of embodiment 9, wherein the predetermined upper voltage magnitude threshold is less than about 3000V for balloons having an outer diameter of 2.5 mm.

20. The intravascular lithotripsy system of embodiment 9, wherein the predetermined lower voltage magnitude threshold is less than about 3000V for balloons having an outer diameter of 3.0 mm.

21. The intravascular lithotripsy system of embodiment 9, wherein the predetermined lower voltage magnitude threshold is less than about 3000V for balloons having an outer diameter of 3.5 mm.

22. The intravascular lithotripsy system of embodiment 9, wherein the predetermined lower voltage magnitude threshold is less than about 3000V for balloons having an outer diameter of 4.0 mm.

23. The intravascular lithotripsy system of embodiment 9, wherein the predetermined upper voltage magnitude threshold is greater than about 3000V for balloons having an outer diameter of 2.5 mm.

24. The intravascular lithotripsy system of embodiment 9, wherein the predetermined lower voltage magnitude threshold is greater than about 3000V for balloons having an outer diameter of 3.0 mm.

25. The intravascular lithotripsy system of embodiment 9, wherein the predetermined lower voltage magnitude threshold is greater than about 3000V for balloons having an outer diameter of 3.5 mm.

26. The intravascular lithotripsy system of embodiment 9, wherein the predetermined lower voltage magnitude threshold is greater than about 3000V for balloons having an outer diameter of 4.0 mm.

27. The intravascular lithotripsy system of any one of embodiments 9 to 26, wherein the predetermined upper voltage magnitude threshold is greater than about 3250V for balloons having an outer diameter of 2.5 mm.

28. The intravascular lithotripsy system of any one of embodiments 9 to 26, wherein the predetermined upper voltage magnitude threshold is greater than about 3250V for balloons having an outer diameter of 3.0 mm.

29. The intravascular lithotripsy system of any one of embodiments 9-26, wherein the predetermined upper voltage magnitude threshold is greater than about 3250V for balloons having an outer diameter of 3.5 mm.

30. The intravascular lithotripsy system of any one of embodiments 9-26, wherein the predetermined upper voltage magnitude threshold is greater than about 3250V for balloons having an outer diameter of 4.0 mm.

31. The intravascular lithotripsy system of any one of the embodiments 1-30, wherein the IVL control system is configured to determine whether the target voltage is not at the predetermined upper voltage magnitude target for a prior executed series of voltage pulses, and to increase the target voltage magnitude by a predetermined amount when the target voltage is determined to not be at the predetermined upper voltage magnitude target.

32. The intravascular lithotripsy system of embodiment 31, wherein the predetermined amount of voltage magnitude increase is within the range of 1 to 250V.

33. The intravascular lithotripsy system of any one of the embodiments 1-32, wherein the target voltage magnitude is increased by 25V when the target voltage is not at the predetermined upper voltage magnitude target for a prior executed series of voltage pulses.

34. The intravascular lithotripsy system of any one of the embodiments 1-32, wherein the target voltage magnitude is increased by more than 25V when the target voltage is not at the predetermined upper voltage magnitude target for a prior executed series of voltage pulses.

35. The intravascular lithotripsy system of any one of the embodiments 1-32, wherein the target voltage magnitude is increased by less than 25V when the target voltage is not at the predetermined upper voltage magnitude target for a prior executed voltage pulse.

36. The intravascular lithotripsy system of any one of the embodiments 1-32, wherein the target voltage magnitude is increased by 25V when the target voltage is not at the predetermined upper voltage magnitude target for a prior executed voltage pulse.

37. The intravascular lithotripsy system of any one of the embodiments 1-32, wherein the target voltage magnitude is increased by more than 25V when the target voltage is not at the predetermined upper voltage magnitude target for a prior executed voltage pulse.

38. The intravascular lithotripsy system of any one of the embodiments 1-32, wherein the target voltage magnitude is increased by less than 25V when the target voltage is not at the predetermined upper voltage magnitude target for a prior executed voltage pulse.

39. The intravascular lithotripsy system of any of the embodiments 1-38, wherein the pressure output over the predetermined maximum number of voltage pulses does not decay or decrease on average more than 0.25 Mpa.

40. The intravascular lithotripsy system of any of the embodiments 1-38, wherein the pressure output across a range of 10 to 300 voltage pulses does not decay or decrease on average more than 0.25 MPa.

41. The intravascular lithotripsy system of any of the embodiments 1-38, wherein the pressure output of the last voltage pulse of the predetermined maximum number of voltage pulses is greater than the pressure output of the first voltage pulse.

42. The intravascular lithotripsy system of any of the embodiments 1-41, wherein a slope of the pressure output of the voltage pulses increases over time.

43. The intravascular lithotripsy system of any of the embodiments 1-40, wherein a slope of the pressure output of the voltage pulses decreases over time.

44. The intravascular lithotripsy system of any of the embodiments 1-40, wherein a slope of the pressure output of the voltage pulses indicates a constant pressure magnitude output across the voltage pulses.

45. The intravascular lithotripsy system of any of the embodiments 1-44, wherein a plurality of a series of voltage pulses are generated.

46. The intravascular lithotripsy system of any of the embodiments 1-45, wherein one or more of the series of voltage pulses comprises 10 voltage pulses.

47. The intravascular lithotripsy system of any of the embodiments 1-45, wherein one or more of the series of voltage pulses comprises more than 10 voltage pulses.

48. The intravascular lithotripsy system of any of the embodiments 1-45, wherein one or more of the series of voltage pulses comprises less than 10 voltage pulses.

49. A method for generating and controlling voltage pulses, comprising:
providing a device according to any one of the embodiments 1-48;
determining the outer diameter of the device's balloon;
establishing an acceptable voltage pulse window comprising a predetermined lower voltage magnitude threshold and a predetermined upper voltage magnitude threshold;
executing a first series of voltage pulses at the predetermined lower voltage magnitude threshold;
increasing the voltage magnitude target by a predetermined amount and execute another series of voltage pulses at the increased voltage magnitude target;
continuing to sequentially increase the voltage magnitude target and executing of a related series of voltage pulses at the sequentially increased voltage magnitude target until the voltage magnitude target equals the predetermined upper voltage magnitude threshold;
determining that a predetermined number of voltage pulses have not been executed; and
continuing executing one or more series of voltage pulses at the upper voltage magnitude threshold until it is determined that predetermined number of voltage pulses have been executed; and
stopping the executing of the voltage pulses.

50. A method for generating and controlling voltage pulses, comprising:
providing a device according to any one of the embodiments 1-48;
determining the outer diameter of the device's balloon;
establishing an acceptable voltage pulse window comprising a predetermined lower voltage magnitude threshold and a predetermined upper voltage magnitude threshold;
executing a first series of voltage pulses at the predetermined lower voltage magnitude threshold;
increasing the voltage magnitude target by a predetermined amount and execute another series of voltage pulses at the increased voltage magnitude target;
continuing to sequentially increase the voltage magnitude target and executing of a related series of voltage pulses at the sequentially increased voltage magnitude target until the voltage magnitude target equals the predetermined upper voltage magnitude threshold;
determining that a predetermined number of voltage pulses have been executed; and
stopping the executing of the voltage pulses.

51. A method for generating and controlling voltage pulses that produce
pressure output that is stable and substantially constant, comprising:
providing a device according to any one of the embodiments 1-48;
determining the outer diameter of the device's balloon;
establishing an acceptable voltage pulse window comprising a predetermined lower voltage magnitude threshold and a predetermined upper voltage magnitude threshold;
executing a first series of voltage pulses at the predetermined lower voltage magnitude threshold;
increasing the voltage magnitude target by a predetermined amount and execute another series of voltage pulses at the increased voltage magnitude target;
continuing to sequentially increase the voltage magnitude target and executing of a related series of voltage pulses at the sequentially increased voltage magnitude target until the voltage magnitude target equals the predetermined upper voltage magnitude threshold; and
producing a pressure output for each voltage pulse comprising a magnitude that is stable and substantially constant.

52. The method of embodiment 51, wherein the pressure output is generated over 10 to at least 300 voltage pulses.

53. A method for generating and controlling voltage pulses that produce pressure output that increases from the first voltage pulse to the last voltage pulse, comprising:
providing a device according to any one of the embodiments 1-48;
determining the outer diameter of the device's balloon;
establishing an acceptable voltage pulse window comprising a predetermined lower voltage magnitude threshold and a predetermined upper voltage magnitude threshold;
executing a first series of voltage pulses at the predetermined lower voltage magnitude threshold;
increasing the voltage magnitude target by a predetermined amount and execute another series of voltage pulses at the increased voltage magnitude target;
continuing to sequentially increase the voltage magnitude target and executing of a related series of voltage pulses at the sequentially increased voltage magnitude target until the voltage magnitude target equals the predetermined upper voltage magnitude threshold; and
producing a pressure output for each voltage pulse comprising a magnitude that increases from the first voltage pulse to the last voltage pulse.

54. The method of embodiment 53, wherein the pressure output is generated over 10 to at least 300 voltage pulses.

55. The method of any one of embodiments 49-54, wherein the voltage pulses are generated at a frequency that is within the range of 1 to 5 pulses/second.

56. The method of embodiment 55, wherein the voltage pulse frequency comprises 2 pulses/second.

57. The method of embodiment 55, wherein the voltage pulse frequency comprises 3 pulses/second.

58. The method of any one of embodiments 49-57, wherein the pressure output of a first balloon comprising an outer diameter is not smaller than the pressure output of a second balloon comprising an outer diameter that is smaller than the outer diameter of the first balloon.

### Exemplary Embodiment Set 2

1. An intravascular lithotripsy system with controlled stable and constant pressure output across a series of voltage pulses, comprising:
at least one set of spaced-apart electrodes for arrangement within a body lumen while disposed within a fluid-fillable member configured to contain conductive fluid therein;
an electric pulse generation system for providing electrical energy to the at least one set of spaced-apart electrodes to generate a plurality of pressure waves for intravascular lithotripsy (IVL) therapy, the electric pulse generation system including an IVL control system comprising a processor configured to execute instructions stored on a memory, and circuitry configured to communicate signals based on operation of the processor, the IVL control system configured to:
generate an initial plurality of voltage pulses comprising an initial series of voltage pulses to the at least one set of spaced-apart electrodes, wherein the magnitude of each voltage pulse in the initial series of voltage pulses comprises a target voltage that is initially set at a predetermined lower voltage magnitude threshold, wherein more than one of the generated plurality of voltage pulses produce a pressure wave,
generate one or more subsequent series of voltage pulses, each subsequent series comprising a plurality of voltage pulses, wherein the target voltage for each subsequent series of voltage pulses is increased by a predetermined amount,
wherein each one of the plurality of the produced pressure waves comprises a pressure magnitude output, and
wherein the IVL control system is configured to control the pressure magnitude output for the plurality of pressure waves such that the pressure magnitude output does not decay or decrease on average more than a predetermined amount across the plurality of pressure waves.

2. An intravascular lithotripsy system with controlled stable and constant pressure output across a series of voltage pulses, comprising:
at least one set of spaced-apart electrodes for arrangement within a body lumen while disposed within a fluid-fillable member configured to contain conductive fluid therein;
an electric pulse generation system for providing electrical energy to the at least one set of electrode to generate a plurality of pressure waves for intravascular lithotripsy (IVL) therapy, the electric pulse generation system including an IVL control system comprising a processor configured to execute instructions stored on a memory, and circuitry configured to communicate signals based on operation of the processor, the IVL control system configured to:
generate an initial plurality of voltage pulses comprising an initial series of voltage pulses to the at least one set of spaced-apart electrodes, wherein the magnitude of each voltage pulse in the initial series of voltage pulses comprises a target voltage that is initially set at a predetermined lower voltage magnitude threshold, wherein more than one of the generated plurality of voltage pulses produce a pressure wave,
generate one or more subsequent series of voltage pulses, each subsequent series comprising a plurality of voltage pulses, wherein the target voltage for each subsequent series of voltage pulses is increased by a predetermined amount,
wherein each one of the plurality of the produced pressure waves comprises a pressure magnitude output, and
wherein the IVL control system is configured to control the target voltage such that the pressure magnitude output does not decay or decrease on average more than a predetermined amount across the plurality of pressure waves.

3. A method for generating and controlling voltage pulses that produce controlled pressure output that is stable and substantially constant across a series of voltage pulses in an intravascular lithotripsy system, comprising:
providing an intravascular lithotripsy system according to embodiment 2;
determining the outer diameter of the intravascular lithotripsy system's balloon;
establishing an acceptable voltage pulse window comprising a predetermined lower voltage magnitude threshold and a predetermined upper voltage magnitude threshold;
executing a first series of voltage pulses at the predetermined lower voltage magnitude threshold;
increasing the voltage magnitude target by a predetermined amount and execute another series of voltage pulses at the increased voltage magnitude target;
continuing to sequentially increase the voltage magnitude target and executing of a related series of voltage pulses at the sequentially increased voltage magnitude target until the voltage magnitude target equals the predetermined upper voltage magnitude threshold; and
producing a plurality of pressure waves, each pressure wave comprising a pressure output comprising a pressure magnitude that is controlled to not decay or decrease on average more than a predetermined amount across the plurality of pressure waves.

4. An intravascular lithotripsy system with controlled pressure output magnitude across a series of voltage pulses, comprising:
at least one set of spaced-apart electrodes for arrangement within a body lumen while disposed within a fluid-fillable member configured to contain conductive fluid therein;
an electric pulse generation system for providing electrical energy to the at least one set of spaced-apart electrodes to generate a plurality of pressure waves for intravascular lithotripsy (IVL) therapy, the electric pulse generation system including an IVL control system comprising a processor configured to execute instructions stored on a memory, and circuitry configured to communicate signals based on operation of the processor, the IVL control system configured to:
generate an initial plurality of voltage pulses comprising an initial series of voltage pulses to the at least one set of spaced-apart electrodes, wherein the magnitude of each voltage pulse in the initial series of voltage pulses comprises a target voltage that is initially set at a predetermined lower voltage magnitude threshold, wherein more than one of the generated plurality of voltage pulses produce a pressure wave,
generate one or more subsequent series of voltage pulses, each subsequent series comprising a plurality of voltage pulses, wherein the target voltage for each subsequent series of voltage pulses is increased by a predetermined amount,
wherein each one of the plurality of the produced pressure waves comprises a pressure magnitude output, and
wherein the IVL control system is configured to control the pressure magnitude output within predetermined upper and lower threshold magnitudes across the plurality of pressure waves.

5. An intravascular lithotripsy system with controlled pressure output magnitude across a series of voltage pulses, comprising:
at least one set of spaced-apart electrodes for arrangement within a body lumen while disposed within a fluid-fillable member configured to contain conductive fluid therein;
an electric pulse generation system for providing electrical energy to the at least one set of spaced-apart electrodes to generate a plurality of pressure waves for intravascular lithotripsy (IVL) therapy, the electric pulse generation system including an IVL control system comprising a processor configured to execute instructions stored on a memory, and circuitry configured to communicate signals based on operation of the processor, the IVL control system configured to:
generate an initial plurality of voltage pulses comprising an initial series of voltage pulses to the at least one set of spaced-apart electrodes, wherein the magnitude of each voltage pulse in the initial series of voltage pulses comprises a target voltage that is initially set at a predetermined lower voltage magnitude threshold, wherein more than one of the generated plurality of voltage pulses produce a pressure wave,
generate one or more subsequent series of voltage pulses, each subsequent series comprising a plurality of voltage pulses, wherein the target voltage for each subsequent series of voltage pulses is increased by a predetermined amount,
wherein each one of the plurality of the produced pressure waves comprises a pressure magnitude output, and
wherein the IVL control system is configured to control the target voltage within predetermined upper and lower thresholds, and further configured to control the resulting pressure wave output within predetermined upper and lower threshold magnitudes across the plurality of pressure waves.

6. A method for controlling pressure output magnitude across a series of voltage pulses generated by an intravascular lithotripsy system, comprising:
providing an intravascular lithotripsy system according to embodiment 5, wherein the fluid-filled member comprises a balloon;
determining the outer diameter of the intravascular lithotripsy system's balloon;
establishing an acceptable voltage pulse window comprising a predetermined lower voltage magnitude threshold and a predetermined upper voltage magnitude threshold;
executing a first series of voltage pulses at the predetermined lower voltage magnitude threshold;
increasing the voltage magnitude target by a predetermined amount and execute another series of voltage pulses at the increased voltage magnitude target;
continuing to sequentially increase the voltage magnitude target and executing of a related series of voltage pulses at the sequentially increased voltage magnitude target until the voltage magnitude target equals the predetermined upper voltage magnitude threshold; and
producing a plurality of pressure waves, each pressure wave comprising a pressure output comprising a pressure magnitude that is controlled to not decay or decrease such that the pressure output across the plurality of pressure waves remains above a predetermined pressure magnitude.

7. An intravascular lithotripsy system with controlled pressure output across a plurality of voltage pulses, comprising:
at least one set of spaced-apart electrodes for arrangement within a body lumen while disposed within a fluid-fillable member configured to contain conductive fluid therein;
an electric pulse generation system for providing electrical energy to the at least one set of spaced-apart electrodes to generate a plurality of pressure waves for intravascular lithotripsy (IVL) therapy, the electric pulse generation system including an IVL control system comprising a processor configured to execute instructions stored on a memory, and circuitry configured to communicate signals based on operation of the processor, the IVL control system configured to:
generate an initial plurality of voltage pulses to the at least one set of electrodes, wherein the magnitude of each voltage pulse in the initial series of voltage pulses comprises a target voltage that is initially set at a predetermined lower voltage magnitude threshold, wherein more than one of the generated plurality of voltage pulses produce a pressure wave,
determine that a predetermined maximum number of voltage pulses have not been executed, and
when the predetermined maximum number of voltage pulses have been determined to not have been executed, sequentially increase the target voltage a predetermined amount and executing related series of voltage pulses until the target voltage is determined to meet a predetermined upper voltage threshold and/or the predetermined maximum number of voltage pulses is determined to have been executed,
wherein each one of the plurality of the produced pressure waves comprises a pressure magnitude output,
wherein the IVL control system is configured to control the pressure magnitude output within a predetermined upper and lower pressure magnitude threshold magnitudes across the plurality of pressure waves, and
terminate the execution of voltage pulses when the predetermined maximum number of voltage pulses have been determined to have been executed.

8. A method for generating and controlling voltage pulses that produce controlled pressure output that is stable and substantially constant across a series of voltage pulses in an intravascular lithotripsy system, comprising:
providing an intravascular lithotripsy system according to embodiment 1, wherein the fluid-filled member comprises a balloon;
determining the outer diameter of the intravascular lithotripsy system's balloon;
establishing an acceptable voltage pulse window comprising a predetermined lower voltage magnitude threshold and a predetermined upper voltage magnitude threshold;
executing a first series of voltage pulses at the predetermined lower voltage magnitude threshold;
increasing the voltage magnitude target by a predetermined amount and execute another series of voltage pulses at the increased voltage magnitude target;
continuing to sequentially increase the voltage magnitude target and executing of a related series of voltage pulses at the sequentially increased voltage magnitude target until the voltage magnitude target equals the predetermined upper voltage magnitude threshold;
producing a plurality of pressure waves, each pressure wave comprising a pressure output comprising a pressure magnitude that is controlled within upper and lower pressure magnitude threshold magnitudes across the plurality of pressure waves;
determining that a predetermined maximum number of voltage pulses have been executed; and
terminating the executing of the voltage pulses.

9. An intravascular lithotripsy system comprising controlled increasing pressure output, comprising:
at least one set of spaced-apart electrodes for arrangement within a body lumen while disposed within a fluid-fillable member configured to contain conductive fluid therein;
an electric pulse generation system for providing electrical energy to the at least one set of spaced-apart electrodes to generate a plurality of pressure waves for intravascular lithotripsy (IVL) therapy, the electric pulse generation system including an IVL control system comprising a processor configured to execute instructions stored on a memory, and circuitry configured to communicate signals based on operation of the processor, the IVL control system configured to:
generate an initial plurality of voltage pulses comprising an initial series of voltage pulses to the at least one set of spaced-apart electrodes,
wherein the magnitude of each voltage pulse in the initial series of voltage pulses comprises a target voltage that is initially set at a predetermined lower voltage magnitude threshold,
wherein more than one of the generated plurality of voltage pulses produce a pressure wave,
generate one or more subsequent series of voltage pulses, each subsequent series comprising a plurality of voltage pulses,
wherein the target voltage for each subsequent series of voltage pulses is increased by a predetermined amount,
wherein each one of the plurality of the produced pressure waves comprises a pressure magnitude output, and
wherein the IVL control system is configured to control the pressure magnitude output for the plurality of pressure waves such that the pressure magnitude output increases across the plurality of pressure waves.

10. An intravascular lithotripsy system comprising controlled increasing pressure output across a series of voltage pulses, comprising:
at least one set of electrodes for arrangement within a body lumen while disposed within a fluid-fillable member configured to contain conductive fluid therein;
an electric pulse generation system for providing electrical energy to the at least one set of spaced-apart electrodes to generate a plurality of pressure waves for intravascular lithotripsy (IVL) therapy, the electric pulse generation system including an IVL control system comprising a processor for executing instructions stored on a memory, and circuitry configured for communication of signals based on operation of the processor, the IVL control system configured to:
generate an initial plurality of voltage pulses comprising an initial series of voltage pulses to the at least one set of spaced-apart electrodes, wherein the magnitude of each voltage pulse in the initial series of voltage pulses comprises a target voltage that is initially set at a predetermined lower voltage magnitude threshold, wherein more than one of the generated plurality of voltage pulses produce a pressure wave,
generate one or more subsequent series of voltage pulses, each subsequent series comprising a plurality of voltage pulses, wherein the target voltage for each subsequent series of voltage pulses is increased by a predetermined amount,
wherein each one of the plurality of the produced pressure waves comprises a pressure magnitude output, and
wherein the IVL control system is configured to control the target voltage such that the pressure magnitude output is controlled to increase within a predetermined pressure magnitude window across the plurality of pressure waves.

11. A method for generating and controlling voltage pulses that produce controlled increasing pressure output in an intravascular lithotripsy system, comprising:
providing an intravascular lithotripsy system according to embodiment 2, wherein the fluid-filled member comprises a balloon;
determining the outer diameter of the intravascular lithotripsy system's balloon;
establishing an acceptable voltage pulse window comprising a predetermined lower voltage magnitude threshold and a predetermined upper voltage magnitude threshold;
executing a first series of more than one voltage pulses at the predetermined lower voltage magnitude threshold;
increasing the voltage magnitude target by a predetermined amount and execute another series of voltage pulses at the increased voltage magnitude target;
continuing to sequentially increase the voltage magnitude target and executing of a related series of voltage pulses at the sequentially increased voltage magnitude target until the voltage magnitude target equals the predetermined upper voltage magnitude threshold; and
producing a plurality of pressure waves, each pressure wave comprising a pressure output comprising a pressure magnitude that is controlled to increase across the plurality of pressure waves.

12. An intravascular lithotripsy system producing controlled decreasing pressure output, comprising:
at least one set of spaced-apart electrodes for arrangement within a body lumen while disposed within a fluid-fillable member configured to contain conductive fluid therein;
an electric pulse generation system for providing electrical energy to the at least one set of spaced-apart electrodes to generate a plurality of pressure waves for intravascular lithotripsy (IVL) therapy, the electric pulse generation system including an IVL control system comprising a processor configured to execute instructions stored on a memory, and circuitry configured to communicate signals based on operation of the processor, the IVL control system configured to:
generate an initial plurality of voltage pulses comprising an initial series of voltage pulses to the at least one set of spaced-apart electrodes, wherein the magnitude of each voltage pulse in the initial series of voltage pulses comprises a target voltage that is initially set at a predetermined lower voltage magnitude threshold, wherein more than one of the generated plurality of voltage pulses produce a pressure wave,
generate one or more subsequent series of voltage pulses, each subsequent series comprising a plurality of voltage pulses, wherein the target voltage for each subsequent series of voltage pulses is increased by a predetermined amount,
wherein each one of the plurality of the produced pressure waves comprises a pressure magnitude output, and
wherein the IVL control system is configured to control the pressure magnitude output for the plurality of pressure waves such that the pressure magnitude output decays or decreases within a predetermined pressure magnitude window across the plurality of pressure waves.

13. An intravascular lithotripsy system producing controlled decreasing pressure output across a series of voltage pulses, comprising:
at least one set of spaced-apart electrodes for arrangement within a body lumen while disposed within a fluid-fillable member configured to contain conductive fluid therein;
an electric pulse generation system for providing electrical energy to the at least one set of spaced-apart electrodes to generate a plurality of pressure waves for intravascular lithotripsy (IVL) therapy, the electric pulse generation system including an IVL control system comprising a processor for executing instructions stored on a memory, and circuitry configured for communication of signals based on operation of the processor, the IVL control system configured to:
generate an initial plurality of voltage pulses comprising an initial series of voltage pulses to the at least one set of electrodes, wherein the magnitude of each voltage pulse in the initial series of voltage pulses comprises a target voltage that is initially set at a predetermined lower voltage magnitude threshold, wherein more than one of the generated plurality of voltage pulses produce a pressure wave,
generate one or more subsequent series of voltage pulses, each subsequent series comprising a plurality of voltage pulses, wherein the target voltage for each subsequent series of voltage pulses is increased by a predetermined amount,
wherein each one of the plurality of the produced pressure waves comprises a pressure magnitude output, and
wherein the IVL control system is configured to control the target voltage such that the pressure magnitude output decreases within a predetermined pressure magnitude window across the plurality of pressure waves.

14. A method for generating and controlling voltage pulses that produce controlled decreasing pressure output in an intravascular lithotripsy system, comprising:
providing an intravascular lithotripsy system according to embodiment 2, wherein the fluid-filled member comprises a balloon;
determining the outer diameter of the intravascular lithotripsy system's balloon;
establishing an acceptable voltage pulse window comprising a predetermined lower voltage magnitude threshold and a predetermined upper voltage magnitude threshold;
executing a first series of more than one voltage pulses at the predetermined lower voltage magnitude threshold;
increasing the voltage magnitude target by a predetermined amount and execute another series of voltage pulses at the increased voltage magnitude target;
continuing to sequentially increase the voltage magnitude target and executing of a related series of voltage pulses at the sequentially increased voltage magnitude target until the voltage magnitude target equals the predetermined upper voltage magnitude threshold; and
producing a plurality of pressure waves, each pressure wave comprising a pressure output comprising a pressure magnitude that is controlled to decrease within a predetermined pressure magnitude window across the plurality of pressure waves.

### Exemplary Embodiment Set 3

1. An IVL system comprising
at least one set of spaced-apart electrodes for arrangement within a body lumen while disposed within a fluid-fillable member configured to contain conductive fluid therein;
an electric pulse generation system for providing electrical energy to the at least one set of spaced-apart electrodes to generate a plurality of pressure waves for intravascular lithotripsy (IVL) therapy, the electric pulse generation system including an IVL control system comprising a processor for executing instructions stored on a memory, and circuitry configured for communication of signals based on operation of the processor, the IVL control system configured to:
generate an initial plurality of voltage pulses comprising at least an initial series of voltage pulses to the at least one set of electrodes, wherein the magnitude of each voltage pulse in the initial series of voltage pulses comprises a target voltage that is initially set at a predetermined lower voltage magnitude threshold, wherein more than one of the generated plurality of voltage pulses produce a pressure wave,
wherein the IVL control system is configured to control the pressure magnitude output across the plurality of pressure waves.

2. The IVL system of embodiment 1, wherein the IVL control system is configured to control the pressure magnitude output is controlled to not decay or decrease more than a predetermined amount across the plurality of pressure waves.

3. The IVL system of embodiment 2, wherein the IVL control system is configured to control the target voltage within predetermined upper and lower thresholds.

4. The IVL system of embodiment 1, wherein the IVL control system is configured to control the pressure magnitude output to remain above a predetermined lower threshold across the plurality of pressure waves.

5. The IVL system of embodiment 4, wherein the IVL control system is configured to control the target voltage within predetermined upper and lower thresholds.

6. The IVL system of embodiment 1, wherein the IVL control system is configured to control the pressure magnitude output to remain within predetermined upper and lower thresholds across the plurality of pressure waves.

7. The IVL system of embodiment 6, wherein the IVL control system is configured to control the target voltage within predetermined upper and lower thresholds.

8. The IVL system of embodiment 1, wherein the IVL control system is configured to control the pressure magnitude output to remain at a substantially constant magnitude across the plurality of pressure waves.

9. The IVL system of embodiment 8, wherein the IVL control system is configured to control the target voltage within predetermined upper and lower thresholds.

10. The IVL system of embodiment 1, wherein the IVL control system is configured to control the pressure magnitude output is controlled to not increase more than a predetermined amount across the plurality of pressure waves.

11. The IVL system of embodiment 10, wherein the IVL control system is configured to control the target voltage within predetermined upper and lower thresholds.

12. The IVL system of embodiment 1, wherein the IVL control system is further configured to terminate the execution of voltage pulses when the predetermined maximum number of voltage pulses have been determined to be executed.

### Exemplary Embodiment Set 4

1. An intravascular lithotripsy system comprising:
at least one set of electrodes for arrangement within a body lumen while disposed within a fluid-fillable member configured to contain conductive fluid therein;
an electric pulse generation system for providing electrical energy to the at least one set of electrode to generate spark for intravascular lithotripsy (IVL) therapy, the electric pulse generation system including an IVL control system comprising a processor for executing instructions stored on a memory, and circuitry configured for communication of signals based on operation of the processor, the IVL control system configured to:
generate an initial series of voltage pulses to the at least one set of electrodes, wherein the magnitude of each voltage pulse in the initial series of voltage pulses comprises a target voltage that is set at a predetermined lower voltage magnitude threshold,
determine whether a threshold parameter is achieved, and
responsive to determination that the threshold parameter is not achieved, increase the voltage magnitude by a predetermined amount, and
generate another series of voltage pulses at the increased voltage magnitude for application to the at least one set of electrodes.

2. The intravascular lithotripsy system of embodiment 1, wherein the IVL control system is configured to continue to determine whether a threshold parameter is achieved after each generated series of voltage pulses.

3. The intravascular lithotripsy system of embodiment 2, wherein the threshold parameter comprises a predetermined upper voltage magnitude threshold.

4. The intravascular lithotripsy system of embodiment 3, wherein the IVL control system is configured to determine if the number of voltage pulses generated does not exceed a predetermined maximum number of voltage pulses.

5. The intravascular lithotripsy system of embodiment 3, wherein the predetermined maximum number of voltage pulses is within a range of 10 to 300 voltage pulses.

6. The intravascular lithotripsy system of embodiment 4, wherein if the IVL control system is configured to determine that the predetermined number of voltage pulses have not been generated, and if the predetermined number of voltage pulses have not been generated, then the IVL control system is further configured to execute another series of voltage pulses at the predetermined upper voltage magnitude threshold.

7. The intravascular lithotripsy system of embodiment 4, wherein if the IVL control system determines that the predetermined number of voltage pulses have been generated, then no further voltage pulses are executed.

8. The intravascular lithotripsy system of any one of embodiments 1-7, wherein the IVL control system is configured to define an acceptable voltage magnitude window comprising the predetermined lower voltage magnitude threshold and the predetermined upper voltage magnitude threshold.

9. The intravascular lithotripsy system of embodiment 8, wherein the acceptable voltage magnitude window is different for balloons of different outer diameters.

10. The intravascular lithotripsy system of embodiment 9, wherein the predetermined lower voltage magnitude threshold is about 2500V for balloons having an outer diameter of 2.5 mm.

11. The intravascular lithotripsy system of embodiment 9, wherein the predetermined lower voltage magnitude threshold comprises about 2500V for balloons having an outer diameter of 3.0 mm.

12. The intravascular lithotripsy system of embodiment 9, wherein the predetermined lower voltage magnitude threshold comprises about 2500V for balloons having an outer diameter of 3.5 mm.

13. The intravascular lithotripsy system of embodiment 9, wherein the predetermined lower voltage magnitude threshold comprises about 2500V for balloons having an outer diameter of 4.0 mm.

14. The intravascular lithotripsy system of embodiment 9, wherein the predetermined lower voltage magnitude threshold is about 3000V for balloons having an outer diameter of 2.5 mm.

15. The intravascular lithotripsy system of embodiment 9, wherein the predetermined lower voltage magnitude threshold comprises about 3000V for balloons having an outer diameter of 3.0 mm.

16. The intravascular lithotripsy system of embodiment 9, wherein the predetermined lower voltage magnitude threshold comprises about 3000V for balloons having an outer diameter of 3.5 mm.

17. The intravascular lithotripsy system of embodiment 9, wherein the predetermined lower voltage magnitude threshold comprises about 3000V for balloons having an outer diameter of 4.0 mm.

18. The intravascular lithotripsy system of embodiment 9, wherein the predetermined lower voltage magnitude threshold is less than about 3000V for balloons having an outer diameter of 2.0 mm.

19. The intravascular lithotripsy system of embodiment 9, wherein the predetermined upper voltage magnitude threshold is less than about 3000V for balloons having an outer diameter of 2.5 mm.

20. The intravascular lithotripsy system of embodiment 9, wherein the predetermined lower voltage magnitude threshold is less than about 3000V for balloons having an outer diameter of 3.0 mm.

21. The intravascular lithotripsy system of embodiment 9, wherein the predetermined lower voltage magnitude threshold is less than about 3000V for balloons having an outer diameter of 3.5 mm.

22. The intravascular lithotripsy system of embodiment 9, wherein the predetermined lower voltage magnitude threshold is less than about 3000V for balloons having an outer diameter of 4.0 mm.

23. The intravascular lithotripsy system of embodiment 9, wherein the predetermined upper voltage magnitude threshold is greater than about 3000V for balloons having an outer diameter of 2.5 mm.

24. The intravascular lithotripsy system of embodiment 9, wherein the predetermined lower voltage magnitude threshold is greater than about 3000V for balloons having an outer diameter of 3.0 mm.

25. The intravascular lithotripsy system of embodiment 9, wherein the predetermined lower voltage magnitude threshold is greater than about 3000V for balloons having an outer diameter of 3.5 mm.

26. The intravascular lithotripsy system of embodiment 9, wherein the predetermined lower voltage magnitude threshold is greater than about 3000V for balloons having an outer diameter of 4.0 mm.

27. The intravascular lithotripsy system of any one of embodiments 9 to 26, wherein the predetermined upper voltage magnitude threshold is greater than about 3250V for balloons having an outer diameter of 2.5 mm.

28. The intravascular lithotripsy system of any one of embodiments 9 to 26, wherein the predetermined upper voltage magnitude threshold is greater than about 3250V for balloons having an outer diameter of 3.0 mm.

29. The intravascular lithotripsy system of any one of embodiments 9-26, wherein the predetermined upper voltage magnitude threshold is greater than about 3250V for balloons having an outer diameter of 3.5 mm.

30. The intravascular lithotripsy system of any one of embodiments 9-26, wherein the predetermined upper voltage magnitude threshold is greater than about 3250V for balloons having an outer diameter of 4.0 mm.

31. The intravascular lithotripsy system of any one of the embodiments 1-30, wherein the IVL control system is configured to determine whether the target voltage is not at the predetermined upper voltage magnitude target for a prior executed series of voltage pulses, and to increase the target voltage magnitude by a predetermined amount when the target voltage is determined to not be at the predetermined upper voltage magnitude target.

32. The intravascular lithotripsy system of embodiment 31, wherein the predetermined amount of voltage magnitude increase is within the range of 1 to 250V.

33. The intravascular lithotripsy system of any one of the embodiments 1-32, wherein the target voltage magnitude is increased by 25V when the target voltage is not at the predetermined upper voltage magnitude target for a prior executed series of voltage pulses.

34. The intravascular lithotripsy system of any one of the embodiments 1-32, wherein the target voltage magnitude is increased by more than 25V when the target voltage is not at the predetermined upper voltage magnitude target for a prior executed series of voltage pulses.

35. The intravascular lithotripsy system of any one of the embodiments 1-32, wherein the target voltage magnitude is increased by less than 25V when the target voltage is not at the predetermined upper voltage magnitude target for a prior executed voltage pulse.

36. The intravascular lithotripsy system of any one of the embodiments 1-32, wherein the target voltage magnitude is increased by 25V when the target voltage is not at the predetermined upper voltage magnitude target for a prior executed voltage pulse.

37. The intravascular lithotripsy system of any one of the embodiments 1-32, wherein the target voltage magnitude is increased by more than 25V when the target voltage is not at the predetermined upper voltage magnitude target for a prior executed voltage pulse.

38. The intravascular lithotripsy system of any one of the embodiments 1-32, wherein the target voltage magnitude is increased by less than 25V when the target voltage is not at the predetermined upper voltage magnitude target for a prior executed voltage pulse.

39. The intravascular lithotripsy system of any of the embodiments 1-38, wherein the pressure output over the predetermined maximum number of voltage pulses does not decay or decrease on average more than 0.25 Mpa.

40. The intravascular lithotripsy system of any of the embodiments 1-38, wherein the pressure output across a range of 10 to 300 voltage pulses does not decay or decrease on average more than 0.25 MPa.

41. The intravascular lithotripsy system of any of the embodiments 1-38, wherein the pressure output of the last voltage pulse of the predetermined maximum number of voltage pulses is greater than the pressure output of the first voltage pulse.

42. The intravascular lithotripsy system of any of the embodiments 1-41, wherein a slope of the pressure output of the voltage pulses increases over time.

43. The intravascular lithotripsy system of any of the embodiments 1-40, wherein a slope of the pressure output of the voltage pulses decreases over time.

44. The intravascular lithotripsy system of any of the embodiments 1-40, wherein a slope of the pressure output of the voltage pulses indicates a constant pressure magnitude output across the voltage pulses.

45. The intravascular lithotripsy system of any of the embodiments 1-44, wherein a plurality of a series of voltage pulses are generated.

46. The intravascular lithotripsy system of any of the embodiments 1-45, wherein one or more of the series of voltage pulses comprises 10 voltage pulses.

47. The intravascular lithotripsy system of any of the embodiments 1-45, wherein one or more of the series of voltage pulses comprises more than 10 voltage pulses.

48. The intravascular lithotripsy system of any of the embodiments 1-45, wherein one or more of the series of voltage pulses comprises less than 10 voltage pulses.

49. A method for generating and controlling voltage pulses, comprising:
providing a device according to any one of the embodiments 1-48, wherein the fluid-fillable member comprises a balloon;
determining the outer diameter of the device's balloon;
establishing an acceptable voltage pulse window comprising a predetermined lower voltage magnitude threshold and a predetermined upper voltage magnitude threshold;
executing a first series of voltage pulses at the predetermined lower voltage magnitude threshold;
increasing the voltage magnitude target by a predetermined amount and execute another series of voltage pulses at the increased voltage magnitude target;
continuing to sequentially increase the voltage magnitude target and executing of a related series of voltage pulses at the sequentially increased voltage magnitude target until the voltage magnitude target equals the predetermined upper voltage magnitude threshold;
determining that a predetermined number of voltage pulses have not been executed; and
continuing executing one or more series of voltage pulses at the upper voltage magnitude threshold until it is determined that predetermined number of voltage pulses have been executed; and
stopping the executing of the voltage pulses.

50. A method for generating and controlling voltage pulses, comprising:
providing a device according to any one of the embodiments 1-48, wherein the fluid-fillable member comprises a balloon;
determining the outer diameter of the device's balloon;
establishing an acceptable voltage pulse window comprising a predetermined lower voltage magnitude threshold and a predetermined upper voltage magnitude threshold;
executing a first series of voltage pulses at the predetermined lower voltage magnitude threshold;
increasing the voltage magnitude target by a predetermined amount and execute another series of voltage pulses at the increased voltage magnitude target;
continuing to sequentially increase the voltage magnitude target and executing of a related series of voltage pulses at the sequentially increased voltage magnitude target until the voltage magnitude target equals the predetermined upper voltage magnitude threshold;
determining that a predetermined number of voltage pulses have been executed; and
stopping the executing of the voltage pulses.

51. A method for generating and controlling voltage pulses that produce pressure output that is stable and substantially constant, comprising:
providing a device according to any one of the embodiments 1-48, wherein the fluid-fillable member comprises a balloon;
determining the outer diameter of the device's balloon;
establishing an acceptable voltage pulse window comprising a predetermined lower voltage magnitude threshold and a predetermined upper voltage magnitude threshold;
executing a first series of voltage pulses at the predetermined lower voltage magnitude threshold;
increasing the voltage magnitude target by a predetermined amount and execute another series of voltage pulses at the increased voltage magnitude target;
continuing to sequentially increase the voltage magnitude target and executing of a related series of voltage pulses at the sequentially increased voltage magnitude target until the voltage magnitude target equals the predetermined upper voltage magnitude threshold; and
producing a pressure output for each voltage pulse comprising a magnitude that is stable and substantially constant.

52. The method of embodiment 51, wherein the pressure output is generated over 10 to at least 300 voltage pulses.

53. A method for generating and controlling voltage pulses that produce pressure output that increases from the first voltage pulse to the last voltage pulse, comprising:
providing a device according to any one of the embodiments 1-48, wherein the fluid-fillable member comprises a balloon;
determining the outer diameter of the device's balloon;
establishing an acceptable voltage pulse window comprising a predetermined lower voltage magnitude threshold and a predetermined upper voltage magnitude threshold;
executing a first series of voltage pulses at the predetermined lower voltage magnitude threshold;
increasing the voltage magnitude target by a predetermined amount and execute another series of voltage pulses at the increased voltage magnitude target;
continuing to sequentially increase the voltage magnitude target and executing of a related series of voltage pulses at the sequentially increased voltage magnitude target until the voltage magnitude target equals the predetermined upper voltage magnitude threshold; and
producing a pressure output for each voltage pulse comprising a magnitude that increases from the first voltage pulse to the last voltage pulse.

54. The method of embodiment 53, wherein the pressure output is generated over 10 to at least 300 voltage pulses.

55. The method of any one of embodiments 49-54, wherein the voltage pulses are generated at a frequency that is within the range of 1 to 5 pulses/second.

56. The method of embodiment 55, wherein the voltage pulse frequency comprises 2 pulses/second.

57. The method of embodiment 55, wherein the voltage pulse frequency comprises 3 pulses/second.

58. The method of any one of embodiments 49-57, wherein the pressure output of a first balloon comprising an outer diameter is not smaller than the pressure output of a second balloon comprising an outer diameter that is smaller than the outer diameter of the first balloon.

### Exemplary Embodiment Set 5

An intravascular lithotripsy system comprising:
a catheter assembly comprising an elongate member defining a lumen and while disposed within a fluid-fillable member configured to contain conductive fluid therein located on a longitudinal region of the elongate member, the catheter assembly configured to inflate the fluid-fillable with the conductive fluid to facilitate IVL therapy; and
at least one set of spaced-apart electrodes for arrangement within the fluid-fillable member for submerging within the IVL fluid medium;
wherein the IVL control system is configured to apply one or more voltage pulses to the at least one spaced-apart electrodes under initial control settings, to determine whether a threshold parameter comprising the maximum number of voltage pulses resulting in an electrical arc between the at least one set of spaced-apart electrodes is achieved under the initial control settings, and to increase at least one of pulse duration and voltage in response to determination that the threshold parameter is not achieved,
wherein the IVL control system is configured to apply the increased at least one of pulse duration and voltage to the at least one set of spaced-apart electrodes.

2. An intravascular lithotripsy system comprising
a catheter assembly comprising an elongate member defining a lumen and while disposed within a fluid-fillable member configured to contain conductive fluid therein located on a longitudinal region of the elongate member, the catheter assembly configured to inflate the fluid-fillable with the conductive fluid to facilitate IVL therapy; at least one set of spaced-apart electrodes for arrangement within the fluid-fillable member for submerging within the conductive fluid; and
an IVL control system comprising a processor for executing instructions stored on memory and circuitry configured to communicate signals based on operation of the processor for providing IVL therapy to a patient,
wherein the IVL control system is configured to generate and apply one or more voltage pulses to the at least one spaced-apart electrodes under initial control settings, to determine whether a threshold parameter comprising the maximum number of voltage pulses resulting in an electrical arc between the at least one set of spaced-apart electrodes is achieved under the initial control settings, and to increase at least one of pulse duration and voltage in response to determination that the threshold parameter is not achieved,
wherein the IVL control system is configured to apply the increased at least one of pulse duration and voltage to the at least one set of spaced-apart electrodes, and
wherein the IVL control system is configured to continue reapplying the increase at least one of pulse duration and voltage to the at least one spaced-apart set of electrodes after the determination that the threshold parameter is not achieved, and
to terminate the generation of voltage pulses when the determination is made that the threshold parameter is achieved.

3. A method of operating an intravascular lithotripsy (IVL) system having a catheter assembly comprising an elongate member defining a lumen and a fluid-fillable member configured to contain conductive fluid and located at a distal region of the elongate member, the catheter assembly configured to inflate the fluid-fillable member with the conductive fluid to facilitate IVL therapy, at least one set of spaced-apart electrodes for arrangement within the fluid-fillable member for submerging within the IVL fluid medium, and an IVL control system comprising a processor configured to execute instructions stored on memory and communications circuitry configured to communicate signals based on operation of the processor for providing IVL therapy to a patient, the method comprising:
generating voltage pulses and applying the generated voltage pulses under initial electrical settings comprising voltage magnitude and duration of application of generated voltage to the at least one set of spaced-apart electrodes;
determining whether a predetermined maximum number of electrical arcs produced between the at least one set of spaced-apart electrodes is achieved under the initial electrical settings;
increasing at least one of pulse duration and voltage of the electrical settings in response to determination that the threshold parameter is not achieved; and
terminating the generating of the voltage pulses in response to determination that the threshold parameter is achieved.

4. An intravascular lithotripsy system comprising:
at least one set of spaced-apart electrodes for arrangement within a body lumen while disposed within a fluid-fillable member configured to contain a conductive fluid;
an electric pulse generation system for providing controlled levels of electrical energy to the at least one set of spaced-apart electrodes to generate spark for intravascular lithotripsy (IVL) therapy, the electric pulse generation system including an IVL control system comprising a processor configured to execute instructions stored on memory and communications circuitry configured to communicate signals based on commands from the processor, the IVL control system including a charge control system for controlling electrical charging of a discharge system,
wherein the charge control system is configured to provide controlled and variable electrical power to the discharge system at different voltage level for consecutive cycles of charge.

5. An intravascular lithotripsy system comprising:
at least one set of spaced-apart electrodes for arrangement within a body lumen while disposed within a fluid-fillable member configured to contain a conductive fluid;
an electric pulse generation system configured to provide controlled electrical energy levels to the at least one set of spaced-apart electrodes to generate spark for intravascular lithotripsy (IVL) therapy, the electric pulse generation system including an IVL control system comprising a processor configured to execute instructions stored on memory and communications circuitry configured to communicate signals based on commands from the processor, the IVL control system including a charge control system for controlling electrical charging of a discharge system,
wherein the discharge system includes an energy storage system and the charge control system is configured to provide controlled variable voltage power to the discharge system at different voltage for consecutive cycles of charge after energy discharge from the energy storage system.

6. An intravascular lithotripsy system comprising:
at least one set of spaced-apart electrodes for arrangement within a body lumen while disposed within a fluid-fillable member configured to contain a conductive fluid;
an electric pulse generation system for providing electrical energy to the at least one set of spaced-apart electrodes to generate spark for intravascular lithotripsy (IVL) therapy, the electric pulse generation system including an IVL control system comprising a processor configured to execute instructions stored on memory and communications circuitry configured to communicate signals based on commands from the processor, wherein the IVL control system is configured to:
assess the stored energy state of an energy storage system of the IVL system to determine stored energy;
compare the assessed stored energy state against a stored threshold value;
deliver a voltage pulse to at least one set of spaced-apart electrodes of the IVL system;
assess the remaining energy state of the energy storage system of the IVL system to determine remaining energy; and
compare the assessed remaining energy state against a stored threshold value.

7. A method of operating an intravascular lithotripsy system, the method comprising:
assessing the stored energy state of an energy storage system of an intravascular lithotripsy (IVL) system to determine stored energy;
delivering a voltage pulse to at least one set of electrodes of the IVL system;
assessing the remaining energy state of the energy storage system of the IVL system to determine remaining energy; and
comparing at least one of the assessed energy states with a stored threshold value to determine the energy of the voltage pulse.

8. A method of operating an intravascular lithotripsy system including at least one set of spaced-apart electrodes, the method comprising:
assessing the stored energy state of an energy storage system of an intravascular lithotripsy (IVL) system to determine stored energy;
delivering a voltage pulse to at least one of the at least one set of spaced-apart electrodes of the IVL system;
assessing the remaining energy state of the energy storage system of the IVL system to determine remaining energy; and
comparing at least one of the assessed energy states with a stored threshold value to determine the energy of the voltage pulse, wherein assessing the energy state to determine stored energy includes determining a voltage level of the energy storage system.

9. An intravascular lithotripsy system comprising:
at least one set of spaced-apart electrodes for arrangement within a body lumen while disposed within a fluid-fillable member configured to contain a conductive fluid;
an electric pulse generation system for providing electrical energy to the at least one set of spaced-apart electrodes to generate spark for intravascular lithotripsy (IVL) therapy, the electric pulse generation system including an IVL control system comprising a processor configured to execute instructions stored on memory and communications circuitry configured to communicate signals based on commands from the processor,
wherein the electric pulse generation system comprises an electrical power system including an AC mains and a DC power storage system,
wherein the AC mains are configured for connection with a AC power receptacle to receive AC power from infrastructure, and the DC power storage system is configured to receive AC power from the AC mains and to convert AC power to DC power for charging a DC power storage device of the DC power storage system, and
wherein the electric pulse generation system is configured to selectively provide power from the AC mains or the DC power storage system for IVL operations.

10. A method for powering a intravascular lithotripsy system, comprising:
providing the intravascular lithotripsy system of embodiment 1;
selecting provision of power from the AC mains; and
powering the intravascular lithotripsy system

11. An intravascular lithotripsy system comprising:
at least one set of electrodes for arrangement within a body lumen while disposed within a fluid-fillable member configured to contain a conductive fluid;
an electric pulse generation system for providing electrical energy to the at least one set of spaced-apart electrodes to generate spark for intravascular lithotripsy (IVL) therapy, the electric pulse generation system including an IVL control system comprising a processor configured to execute instructions stored on memory and communications circuitry configured to communicate signals based on commands from the processor, wherein the IVL control system is configured to:
assess the stored energy state of an energy storage system of the IVL system to determine stored energy;
deliver a voltage pulse to at least one set of electrodes of the IVL system;
assess the remaining energy state of the energy storage system of the IVL system to determine remaining energy; and
compare the assessed energy states to determine the energy of the voltage pulse.

12. A method of operating an intravascular lithotripsy system, the method comprising:
assessing the stored energy state of an energy storage system of an intravascular lithotripsy (IVL) system to determine stored energy;
delivering a voltage pulse to at least one set of spaced-apart electrodes of the IVL system;
assessing the remaining energy state of the energy storage system of the IVL system to determine remaining energy; and
comparing the energy states assessed to determine the energy of the voltage pulse.

13. A method of operating an intravascular lithotripsy system, the method comprising:
assessing the stored energy state of an energy storage system of an intravascular lithotripsy (IVL) system to determine stored energy;
delivering a voltage pulse to at least one set of spaced-apart electrodes of the IVL system;
assessing the remaining energy state of the energy storage system of the IVL system to determine remaining energy; and
comparing the energy states assessed to determine the energy of the voltage pulse, wherein assessing the energy state to determine stored energy includes determining a voltage level of the energy storage system.

14. An intravascular lithotripsy system comprising:
at least one set of spaced-apart electrodes for arrangement within a body lumen while disposed within a fluid-fillable member configured to contain a conductive fluid;
an electric pulse generation system for providing electrical energy to the at least one set of spaced-apart electrodes to generate spark for intravascular lithotripsy (IVL) therapy, the electric pulse generation system including an IVL control system comprising a processor configured to execute instructions stored on memory and communications circuitry configured to communicate signals based on commands from the processor,
wherein the IVL control system including an adjustable energy storage system configured to selective adjust the electrical energy to be applied to the at least one set of spaced-apart electrodes for IVL therapy.

15. A method of operating an intravascular lithotripsy (IVL) system, the method comprising:
applying a voltage pulse to at least one set of electrodes;
determining to adjust a stored energy level of the IVL system;
applying another voltage pulse to the at least one set of electrodes using an energy level different from the voltage pulse.

### Exemplary Embodiment Set 6

1. An intravascular lithotripsy system comprising:
at least one set of electrodes for arrangement within a body lumen while disposed within a fluid-fillable member configured to contain a conductive fluid;
an electric pulse generation system for providing electrical energy to the at least one set of electrode to generate spark for intravascular lithotripsy (IVL) therapy, the electric pulse generation system including an IVL control system comprising a processor for executing instructions stored on memory and circuitry configured for communication of signals based on operation of the processor, the IVL control system configured to:
apply initial electrical energy to the at least one set of electrodes,
determine whether a threshold parameter is achieved, and
responsive to determination that the threshold parameter is not achieved, increase a duration of a pulse of electrical energy for application to the at least one set of electrodes.

2. The system of embodiment 1, wherein the IVL control system is configured to re-apply initial electrical energy to the at least one set of electrodes, responsive to determination that the threshold parameter is achieved.

3. The system of embodiment 2, wherein the threshold parameter is a value of current for achieving sufficient spark for IVL therapy.

4. The system of embodiment 3, wherein the value of current is about 20 amperes.

5. The system of embodiment 1, wherein the IVL control system is configured to repeat applying of electrical energy to the at least set of electrodes with the increased duration of the pulse of electrical energy.

6. The system of embodiment 5, wherein the IVL control system is configured to determine whether the threshold parameter is achieved under the repeat applying of electrical energy.

7. The system of embodiment 6, wherein the IVL control system is configured to re-apply the electrical energy to the at least one set of electrodes with the increased duration of the pulse of electrical energy, responsive to determination that the threshold parameter is achieved under the repeat applying of electrical energy.

8. The system of embodiment 5, wherein the threshold parameter remains the same value under the initial applying and the repeat applying.

9. The system of embodiment 5, wherein the IVL control system is configured to determine whether a maximum duration is achieved.

10. The system of embodiment 9, wherein, responsive to determination that the maximum duration is achieved, the IVL control system is configured to increase the voltage of the electrical energy applied.

11. The system of embodiment 10, wherein the IVL control system is configured to re-apply the electrical energy to the at least one set of electrodes with the increased voltage.

12. The system of embodiment 10, wherein re-applying the electrical energy to the at least one set of electrodes with the increased voltage includes adjusting the duration as presently selected.

13. The system of embodiment 12, wherein adjusting the duration as presently selected includes reducing the duration.

14. The system of embodiment 13, wherein adjusting the duration as presently selected includes resetting the duration as presently selected equal to duration under the initial electrical energy.

15. The system of embodiment 10, wherein the IVL control system is configured to determine whether a maximum voltage is achieved, and to re-apply the electrical energy to the at least one set of electrodes with the increased voltage responsive to determination that the maximum voltage is not achieved.

16. The system of embodiment 9, wherein the maximum duration remains the same value under re-applying.

17. The system of embodiment 1, wherein applying of electrical energy comprises delivery of a voltage pulse having a voltage and duration.

18. The system of embodiment 17, wherein an initial duration of the initial electrical energy delivered to the at least one electrodes is a voltage pulse having duration within the range of about 0.1 microseconds to about 2 microseconds.

19. The system of embodiment 17, wherein an initial voltage of the initial electrical energy delivered to the at least one electrodes is a voltage pulse having voltage within the range of about 500 volts to about 4000 volts.

20. A method of operating an intravascular lithotripsy system, the method comprising:
applying initial electrical energy to at least one set of electrodes of an intravascular lithotripsy (IVL) system, wherein the at least one set of electrodes is submerged within a conductive fluid contained by a fluid-fillable member;
determining whether a threshold parameter is achieved; and
responsive to determination that the threshold parameter is not achieved, increasing a duration of a pulse of electrical energy for application to the at least one set of electrodes.

21. The method of embodiment 20, further comprising re-applying initial electrical energy to the at least one set of electrodes, responsive to determination that the threshold parameter is achieved.

22. The method of embodiment 21, wherein the threshold parameter is a value of current for achieving sufficient spark for IVL therapy.

23. The method of embodiment 22, wherein the value of current is about 20 amperes.

24. The method of embodiment 20, further comprising repeating applying of electrical energy to the at least set of electrodes with the increased duration of the pulse of electrical energy.

25. The method of embodiment 24, further comprising determining whether the threshold parameter is achieved under the repeat applying of electrical energy.

26. The method of embodiment 25, further comprising re-applying the electrical energy to the at least one set of electrodes with the increased duration of the pulse of electrical energy, responsive to determination that the threshold parameter is achieved under the repeat applying of electrical energy.

27. The method of embodiment 25, wherein the threshold parameter remains the same value under the initial applying and the repeat applying.

28. The method of embodiment 25, further comprising determining whether a maximum duration is achieved.

29. The method of embodiment 28, further comprising increasing the voltage of the electrical energy applied, responsive to determination that the maximum duration is achieved.

30. The method of embodiment 29, further comprising re-applying the electrical energy to the at least one set of electrodes with the increased voltage.

31. The method of embodiment 30, wherein re-applying the electrical energy to the at least one set of electrodes with the increased voltage includes adjusting the duration as presently selected.

32. The method of embodiment 31, wherein adjusting the duration as presently selected includes reducing the duration.

33. The method of embodiment 32, wherein adjusting the duration as presently selected includes resetting the duration as presently selected equal to duration under the initial electrical energy.

34. The method of embodiment 29, further comprising determining whether a maximum voltage is achieved, re-applying the electrical energy to the at least one set of electrodes with the increased voltage responsive to determination that the maximum voltage is not achieved.

35. The method of embodiment 20, wherein applying of electrical energy comprises delivery of a voltage pulse having a voltage and duration.

36. The system of embodiment 35, wherein an initial duration of the initial electrical energy delivered to the at least one electrodes is a voltage pulse having duration within the range of about 0.1 microseconds to about 2 microseconds.

37. The system of embodiment 35, wherein an initial voltage of the initial electrical energy delivered to the at least one electrodes is a voltage pulse having voltage within the range of about 500 volts to about 4000 volts.

38. An intravascular lithotripsy system comprising
a catheter assembly comprising an elongate member defining a lumen and a fluid-fillable member configured to contain a conductive fluid and located at or near a longitudinal end of the elongate member, the catheter assembly configured to fill the fluid-fillable member with the conductive fluid to facilitate IVL therapy;
at least one set of electrodes for arrangement within the fluid-fillable member for submerging the at least one set of electrodes within the conductive fluid;
an IVL therapy control system comprising a processor for executing instructions stored on memory and circuitry configured for communication of signals based on operation of the processor for providing IVL therapy to a patient, the IVL therapy control system configured to apply electrical energy having initial settings, to determine whether a threshold parameter is achieved under the electrical settings, and to increase at least one of pulse duration and voltage in response to determination that the threshold parameter is not achieved.

39. A method of operating an intravascular lithotripsy (IVL) system having a catheter assembly comprising an elongate member defining a lumen and a fluid-fillable member configured to contain a conductive fluid and located at or near a longitudinal end of the elongate member, the catheter assembly configured to inflate the fluid-fillable member with the conductive fluid to facilitate IVL therapy, at least one set of electrodes for arrangement within the fluid-fillable member for submerging within the IVL fluid medium, and an IVL therapy control system comprising a processor for executing instructions stored on memory and communications circuitry configured for communication of signals based on operation of the processor for providing IVL therapy to a patient, the method comprising:
applying electrical energy having electrical settings including initial settings;
determining whether a threshold parameter is achieved under the applied electrical settings; and
increasing at least one of pulse duration and voltage of the electrical settings in response to determination that the threshold parameter is not achieved.

40. An intravascular lithotripsy system comprising:
at least one set of electrodes for arrangement within a body lumen while disposed within a fluid-fillable member configured to contain a conductive fluid;
an electric pulse generation system for providing electrical energy to the at least one set of electrode to generate spark for intravascular lithotripsy (IVL) therapy, the electric pulse generation system including an IVL control system comprising a processor for executing instructions stored on memory and communications circuitry configured for communication of signals based on commands from the processor, IVL control system including an adjustable energy storage system for selectively adjusting the stored energy applied for providing electrical energy to the at least one set of electrodes for IVL therapy.

41. The system of embodiment 40, wherein the electric pulse generation system includes a relay system for selectively connecting a number of energy storage elements of the energy storage system for discharge to provide electrical energy to the at least one set of electrodes.

42. The system of embodiment 41, wherein in an engaged arrangement of the relay system all of the number of energy storage elements are connected to discharge to provide electrical energy to the at least one set of electrodes.

43. The system of embodiment 42, wherein in a disengaged arrangement of the relay system fewer than all of the number of the energy storage elements are connected to discharge to provide electrical energy to the at least one set of electrodes.

44. The system of embodiment 43, wherein the electrical energy provided to the at least one set of electrodes is greater under the one arrangement of the relay system than under the other arrangement of the relay system.

45. The system of embodiment 43, wherein in one pulse of electrical energy to the electrodes the relay system is in either the engaged or disengaged arrangement, and in another pulse of the electrical energy to the electrodes the relay system is in the disengaged arrangement.

46. A method of operating an intravascular lithotripsy (IVL) system, the method comprising:
applying a first voltage pulse to at least one set of electrodes;
determining to adjust a stored energy level of the IVL system;
applying another voltage pulse to the at least one set of electrodes using a stored energy magnitude or level different from the first voltage pulse.

47. The method of embodiment 46, wherein applying the voltage pulse includes operating an adjustable energy storage system at a first stored energy level to apply electrical energy.

48. The method of embodiment 47, wherein applying the other voltage pulse includes operating the adjustable energy storage system at a second stored energy level to apply electrical energy.

49. The method of embodiment 48, wherein the second stored energy level is greater than the first stored energy level.

50. The method of embodiment 48, wherein the second stored energy level is less than the first stored energy level.

51. The method of embodiment 48, wherein applying the voltage pulse includes arranging a relay system to connect one or more stored energy elements of the adjustable energy storage system.

52. The method of embodiment 51, wherein arranging the relay system to connect the one or more stored energy elements of the adjustable energy storage system includes a maximum number of stored energy elements of the adjustable energy storage system.

53. The method of embodiment 51, wherein arranging the relay system to connect the one or more stored energy elements of the adjustable energy storage system includes less than a maximum number of stored energy elements of the adjustable energy storage system.

54. The method of embodiment 48, wherein applying the other voltage pulse includes arranging a relay system to connect additional stored energy elements of the adjustable energy storage system.

55. The method of embodiment 54, wherein arranging the relay system to connect the additional stored energy elements of the adjustable energy storage system includes a maximum number of stored energy elements of the adjustable energy storage system.

56. The method of embodiment 54, wherein arranging the relay system to connect the additional stored energy elements of the adjustable energy storage system includes less than a maximum number of the stored energy elements of the adjustable energy storage system.

57. The method of embodiment 47, wherein applying the voltage pulse includes arranging a relay system to connect a number of the stored energy elements of the adjustable energy storage system.

58. The method of embodiment 57, wherein arranging the relay system to connect the number of the stored energy elements of the adjustable energy storage system includes a maximum number of the stored energy elements of the adjustable energy storage system.

59. The method of embodiment 58, wherein applying the other voltage pulse includes operating the adjustable energy storage system at another stored energy magnitude to apply electrical energy.

60. The method of embodiment 57, wherein arranging the relay system to connect the number of the stored energy elements of the adjustable energy storage system includes less than a maximum number of the stored energy elements of the adjustable energy storage system.

61. The method of embodiment 60, wherein applying the other voltage pulse includes operating the adjustable energy storage system at another stored energy magnitude to apply electrical energy.

62. An intravascular lithotripsy system comprising:
at least one set of spaced-apart electrodes for arrangement within a body lumen while disposed within a fluid-fillable member configured to contain a conductive fluid;
an electric pulse generation system for providing electrical energy to the at least one set of spaced-apart electrodes to generate spark for intravascular lithotripsy (IVL) therapy, the electric pulse generation system including an IVL control system comprising a processor for executing instructions stored on memory and communications circuitry configured for communication of signals based on commands from the processor, the IVL control system including a charge control system for controlling electrical charging of a discharge system.

63. The system of embodiment 62, wherein the charge control system includes low voltage power regulator arranged to receive and control low voltage power for output for conversion to high voltage power.

64. The system of embodiment 63, wherein the charge control system receives a modulated control signal for controlling output of low voltage power for conversion to high voltage power.

65. The system of embodiment 63, wherein the charge control system is arranged to apply the modulated control signal with feedback of the output of low voltage power.

66. The system of embodiment 63, wherein the modulated control signal is a high frequency pulse width modulated signal.

67. The system of embodiment 61, wherein the charge control system includes a converter arranged to receive low voltage power and to convert low voltage power to high voltage power for communication to the discharge system.

68. The system of embodiment 67, wherein the converter is a DC-DC converter.

69. The system of embodiment 62, wherein the charge control system is configured to provide variable voltage power to the discharge system at different voltage level for consecutive cycles of charge.

70. The system of embodiment 62, wherein the discharge system includes a energy storage system and the charge control system is configured to provide variable voltage power to the discharge system at different voltage for consecutive cycles of charge after stored energy discharge.

71. An intravascular lithotripsy system comprising:
at least one set of spaced-apart electrodes for arrangement within a body lumen while disposed within a fluid-fillable member configured to contain a conductive fluid;
an electric pulse generation system for providing electrical energy to the at least one set of spaced-apart electrodes to generate spark for intravascular lithotripsy (IVL) therapy, the electric pulse generation system including an IVL control system comprising a processor for executing instructions stored on memory and communications circuitry configured for communication of signals based on commands from the processor, the IVL control system including an electrical discharge system for selectively communicating high voltage discharge to the at least one set of electrodes.

72. The system of embodiment 71, wherein the electrical discharge system comprises a discharge switching system operable to modulate communication of high voltage power to the at least one set of electrodes for IVL therapy.

73. The system of embodiment 72, wherein the discharge switching system includes at least one gate-controlled switch operable by processor signal to selectively permit communication of high voltage power to the at least one set of electrodes for IVL therapy.

74. The system of embodiment 73, where the at least one gate controlled switch is an insulated gate bipolar transistor.

75. The system of embodiment 73, where the at least one gate controlled switch is selectively operable in a permissive state permitting communication of high voltage power to the at least one set of electrodes for IVL therapy, and a non-permissive state blocking against communication of high voltage power to the at least one set of electrodes for IVL therapy.

76. The system of embodiment 71, wherein the electrical discharge system comprises an energy storage system for charging of high voltage energy for discharge to the at least one set of electrodes.

77. The system of embodiment 76, wherein the electrical discharge system comprises a discharge switching system operable to modulate communication of high voltage power from the energy storage system to the at least one set of electrodes for IVL therapy.

78. A method of operating an intravascular lithotripsy system, the method comprising:
assessing the stored energy state of a energy storage system of an intravascular lithotripsy (IVL) system to determine stored energy;
delivering a voltage pulse to at least one set of electrodes of the IVL system;
assessing the remaining energy state of the energy storage system of the IVL system to determine remaining energy; and
comparing the energy states assessed to determine the energy of the voltage pulse.

79. The method of embodiment 78, wherein assessing the energy state to determine stored energy includes determining a voltage level of the energy storage system.

80. The method of embodiment 79, wherein assessing the energy state to determine stored energy includes determining a level of energy stored within the energy storage system based on the voltage level of the energy storage system.

81. The method of embodiment 78, wherein assessing the energy state to determine remaining energy includes determining a voltage level of the energy storage system.

82. The method of embodiment 81, wherein assessing the energy state to determine remaining energy includes determining a level of energy remaining within the energy storage system based on the voltage level of the energy storage system.

83. The method of embodiment 78, wherein comparing the energy states assessed includes determining the difference between the stored and remaining energy states of the energy storage system as a discharged energy.

84. The method of embodiment 83, wherein comparing the energy states assessed includes comparing the discharged energy with a threshold value.

85. The method of embodiment 84, wherein responsive to a determination that the discharged energy is equal to or greater than the threshold value, determining that sufficient spark has been generated.

86. The method of embodiment 84, wherein responsive to a determination that the discharged energy is less than the threshold value, determining that insufficient spark has been generated.

87. The method of embodiment 78, further comprising determining parameters of a further voltage pulse based on the comparison of energy states.

88. The method of embodiment 87, wherein determining parameters of the further voltage pulse includes, responsive to determination that the voltage pulse generated sufficient spark, maintaining one or more parameters of the voltage pulse for the further voltage pulse.

89. The method of embodiment 88, further comprising delivering the further voltage pulse based on the one or more maintained parameters.

90. The method of embodiment 89, repeating assessing the remaining energy state of the energy storage system of the IVL system to determine remaining energy, after delivery of the further voltage pulse.

91. The method of embodiment 87, wherein determining parameters of the further voltage pulse includes, responsive to determination that the voltage pulse generated insufficient spark, changing one or more parameters of the voltage pulse for the further voltage spark.

92. The method of embodiment 91, wherein changing one or more parameters includes changing duration of pulse for the further voltage pulse.

93. The method of embodiment 92, wherein changing duration includes increasing duration of the further voltage pulse greater than a duration of the voltage pulse.

94. The method of embodiment 92, further comprising delivering the further voltage pulse based on the one or more changed parameters.

95. The method of embodiment 94, repeating assessing the remaining energy state of the energy storage system of the IVL system to determine remaining energy, after delivery of the further voltage pulse.

96. The method of embodiment 78, wherein assessing the energy state to determine stored energy includes assessing the energy state of the energy storage system after an earlier voltage pulse has been delivered.

97. An intravascular lithotripsy system comprising:
at least one set of spaced-apart electrodes for arrangement within a body lumen while disposed within a fluid-fillable member configured to contain a conductive fluid;
an electric pulse generation system for providing electrical energy to the at least one set of spaced-apart electrodes to generate spark for intravascular lithotripsy (IVL) therapy, the electric pulse generation system including an IVL control system comprising a processor for executing instructions stored on memory and communications circuitry configured for communication of signals based on commands from the processor, wherein the IVL control system is configured to:
   assess the stored energy state of a energy storage system of the IVL system to determine stored energy;
   deliver a voltage pulse to at least one set of electrodes of the IVL system;
   assess the remaining energy state of the energy storage system of the IVL system to determine remaining energy; and
   compare the energy states assessed to determine the energy of the voltage pulse.

98. The system of embodiment 97, wherein assessing the energy state to determine stored energy includes determining a voltage level of the energy storage system.

99. The system of embodiment 98, wherein assessing the energy state to determine stored energy includes determining a level of energy stored within the energy storage system based on the voltage level of the energy storage system.

100. The system of embodiment 97, wherein assessing the energy state to determine remaining energy includes determining a voltage level of the energy storage system.

101. The system of embodiment 100, wherein assessing the energy state to determine remaining energy includes determining an energy remaining within the energy storage system based on the voltage level of the energy storage system.

102. The system of embodiment 97, wherein comparing the energy states assessed includes determining the difference between the stored and remaining energy states of the energy storage system as a discharged energy.

103. The system of embodiment 102, wherein comparing the energy states assessed includes comparing the discharged energy with a threshold value.

104. The system of embodiment 103, wherein responsive to a determination that the discharged energy is equal to or greater than the threshold value, determining that sufficient spark has been generated.

105. The system of embodiment 103, wherein responsive to a determination that the discharged energy is less than the threshold value, determining that insufficient spark has been generated.

106. The system of embodiment 97, further comprising determining parameters of a further voltage pulse based on the comparison of energy states.

107. The system of embodiment 106, wherein determining parameters of the further voltage pulse includes, responsive to determination that the voltage pulse generated sufficient spark, maintaining one or more parameters of the further voltage pulse.

108. The system of embodiment 107, further comprising delivering the further voltage pulse based on the one or more maintained parameters.

109. The system of embodiment 108, repeating assessing the remaining energy state of the energy storage system of the IVL system to determine remaining energy, after delivery the further voltage pulse.

110. The method of embodiment 106, wherein determining parameters of the further voltage pulse includes, responsive to determination that the voltage pulse generated insufficient spark, changing one or more parameters of the voltage pulse for the further voltage spark.

111. The method of embodiment 110, wherein changing one or more parameters includes changing duration of pulse for the further voltage pulse.

112. The method of embodiment 111, wherein changing duration includes increasing duration of the further voltage pulse greater than a duration of the voltage pulse.

113. The method of embodiment 111, further comprising delivering the further voltage pulse based on the one or more changed parameters.

114. The method of embodiment 113, repeating assessing the remaining energy state of the energy storage system of the IVL system to determine remaining energy, after delivery of the further voltage pulse.

115. An intravascular lithotripsy system comprising:
at least one set of spaced-apart electrodes for arrangement within a body lumen while disposed within a fluid-fillable member configured to contain a conductive fluid;
an electric pulse generation system for providing electrical energy to the at least one set of spaced-apart electrodes to generate spark for intravascular lithotripsy (IVL) therapy, the electric pulse generation system including an IVL control system comprising a processor for executing instructions stored on memory and communications circuitry configured for communication of signals based on commands from the processor, wherein the electric pulse generation system comprises an electrical power system including an AC mains and a DC power storage system, wherein the AC mains are configured for connection with a AC power receptacle to receive AC power from infrastructure, and the DC power storage system is configured to receive AC power from the AC mains and to convert AC power to DC power for charging a DC power storage device of the DC power storage system,
wherein the electric pulse generation system is configured to selectively provide power from the AC mains or the DC power storage system for IVL operations.

116. The system of embodiment 115, wherein IVL operations includes high voltage pulse provided to the at least one set of electrodes.

117. The system of embodiment 115, wherein the DC power storage device comprises a chemical battery.

118. The system of embodiment 115, wherein the electrical power system is configured to selectively provide power for IVL operations from stored power of the DC power storage device, from the DC power storage device while connected to receive charging power from the AC mains, or directly from the AC mains converted to DC power without the DC power storage device.

The description of devices, systems, and method and related applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

What is claimed is:

1. An intravascular lithotripsy (“IVL”) console, the console comprising:
a power plug configured to be coupled to a mains power source (e.g., an outlet), the power plug extending outside of an enclosure enclosing internal console circuitry;
one or more conductors coupled to the power plug;
a first isolation barrier coupled to the one or more conductors, the isolation barrier configured to electrically, according to at least a predetermined dielectric strength, isolate power from the mains power source from portions of the internal console circuitry;
wherein the internal console circuitry comprises low-voltage control circuitry and high-voltage power circuitry, wherein the high-voltage power circuitry is configured to generate a high-voltage power source for supplying high-voltage pulses to emitters in a catheter couplable to the console;
a second isolation barrier between the low-voltage control circuitry and the high-voltage power circuitry, the second isolation barrier configured to couple a first processor in the low-voltage control circuitry, configured to issue commands to the high-voltage power circuitry, to a second processor in the high-voltage power circuitry, the second processor configured to receive commands from the first processor through the isolation barrier and to perform actions to cause the high-voltage power circuitry to deliver high power pulses to emitters in the catheter; and
wherein the first isolation barrier and second isolation barrier are configured such that the power plug can be coupled to the mains power source and supply power to the circuitry in the enclosure while the high-voltage power circuitry is delivering high voltage pulses to the one or more emitters in the catheter.

2. The IVL console of claim 1, wherein the console is adapted to be associated with an expandable balloon having at least two electrodes for creating a spark.

3. The IVL console of claim 1, wherein the second isolation barrier comprises magnetic coupling to achieve isolation.

4. The IVL console of claim 1, wherein the second isolation barrier comprises optical coupling to achieve isolation.

5. The IVL console of claim 1, further comprising the enclosure, the enclosure housing the internal console circuitry and providing physical protection and electromagnetic shielding for sensitive electronic components.

6. The IVL console of claim 1, further comprising at least one input interface allowing an operator to communicate with and control the console, which is electrically isolated from high-voltage sections of the IVL console.

7. The IVL console of claim 1, wherein the first isolation barrier comprises a transformer.

8. The IVL console of claim 1, wherein the system is configured to split the mains power to charge a battery and power the IVL console.

9. A method for operating an intravascular lithotripsy (IVL) console, the method comprising:
coupling a power plug to a mains power source, the power plug extending outside of an enclosure enclosing internal console circuitry;
conducting power from the mains power source through one or more conductors coupled to the power plug;
electrically isolating power from the mains power source from portions of the internal console circuitry using a first isolation barrier coupled to the one or more conductors, the isolation barrier configured to isolate power according to at least a predetermined dielectric strength;
generating a high-voltage power source using high-voltage power circuitry within the internal console circuitry, the high-voltage power circuitry configured to supply high voltage pulses to emitters in a catheter couplable to the console;
issuing commands from a first processor in low-voltage control circuitry to a second processor in the high-voltage power circuitry through a second isolation barrier, the second isolation barrier configured to couple the first processor to the second processor;
performing actions by the second processor to cause the high-voltage power circuitry to deliver high voltage pulses to emitters in the catheter;
maintaining electrical isolation between the low-voltage control circuitry and the high-voltage power circuitry using the second isolation barrier;
supplying power to the internal console circuitry while the high-voltage power circuitry is delivering high voltage pulses to the emitters in the catheter.

10. The method of claim 9, further comprising associating the console with an expandable balloon having at least two electrodes for creating a spark.

11. The method of claim 9, wherein the second isolation barrier comprises magnetic coupling to achieve isolation.

12. The method of claim 9, wherein the second isolation barrier comprises optical coupling to achieve isolation.

13. The method of claim 9, further comprising housing the internal console circuitry within an enclosure that provides physical protection and electromagnetic shielding for sensitive electronic components.

14. The method of claim 9, further comprising allowing an operator to communicate with and control the console using at least one input interface which is electrically isolated from high-voltage sections of the IVL console.

15. The method of claim 9, wherein the first isolation barrier comprises a transformer.

16. An intravascular lithotripsy (IVL) console comprising:
a power plug configured to couple to a mains power source;
a first isolation barrier coupled to one or more conductors from the power plug, the first isolation barrier configured to electrically isolate mains power from internal console circuitry according to at least a predetermined dielectric strength;
low-voltage control circuitry including a first processor;
high-voltage power circuitry including a second processor and a discharge system configured to generate high-voltage pulses; and
a second isolation barrier disposed between the low-voltage control circuitry and the high-voltage power circuitry, the second isolation barrier configured to communicatively couple the first processor to the second processor while electrically isolating the low-voltage control circuitry from the high-voltage power circuitry;
wherein the first and second isolation barriers collectively enable the console to receive power from the mains power source and simultaneously deliver high-voltage pulses from the high-voltage power circuitry while maintaining electrical isolation of the low-voltage control circuitry from hazardous voltages.

17. The console of claim 16, wherein the first isolation barrier comprises a transformer configured to provide galvanic isolation from the mains supply.

18. The console of claim 16, wherein the second isolation barrier comprises at least one magnetic digital isolator.

19. The console of claim 16, wherein the second isolation barrier comprises at least one optical digital isolator.

20. The console of claim 16, wherein the low-voltage control circuitry and the high-voltage power circuitry are mounted on physically separate circuit boards separated by the second isolation barrier.
